(19) United States
(12) Patent Application Publication
(10) Pub. No.: US 2004/0209310 A1
(43) Pub. Date: Oct. 21, 2004

(54) Ligands that bind to the amyloid-beta precursor peptide and related molecules and uses thereof

(76) Inventors: Thomas C. Sudhof, Dallas, TX (US); Angela Ho, Dallas, TX (US)

Correspondence Address: BAKER & BOTTS, 30 ROCKEFELLER PLAZA, NEW YORK, NY, 10112

(21) Appl. No.: 10/793,435

(22) Filed: Mar. 3, 2004

Related U.S. Application Data
(60) Provisional application No. 60/451,574, filed on Mar. 3, 2003.
(60) Provisional application No. 60/544,669, filed on Feb. 13, 2004.

(51) Int. Cl. G01N 33/53; C07K 16/18

(52) U.S. Cl. 435/7.1; 530/391.1

(57) ABSTRACT

The present invention provides a composition comprising an isolated F-spondin polypeptide specifically bound to an APP or APLP polypeptide. The present invention further provides a composition comprising an isolated neurexin polypeptide specifically bound to an APP or APLP polypeptide. Also provided are methods of screening for modulators of the binding of an APP or an APLP polypeptide by F-spondin or neurexin proteins. Modulators of the binding of an APP or an APLP polypeptide by F-spondin or neurexin proteins may be useful in the treatment or prevention of AD.

### CROSS REFERENCE TO RELATED APPLICATIONS

This application is claims benefit of U.S. Provisional Patent Application Ser. No. 60/451,574, filed Mar. 3, 2003, and U.S. Provisional Patent Application Ser. No. 60/544,669, filed Feb. 13, 2004, the contents of which are incorporated by reference herein in their entireties.

### STATEMENT REGARDING FEDERALLY SPONSORED RESEARCH

The subject matter described herein was supported in part by National Institutes of Health Grant F32-AG05844, so that the United States Government has certain rights herein.

### FIELD OF THE INVENTION

The present invention relates to the identification of specific interactions between certain extracellular domains of the amyloid-β precursor protein (APP) or the APP-like proteins (APLP1 and APLP2) and F-spondin or neurexin proteins. The identification of F-spondin and neurexin proteins as endogenous ligands of APP and APLPs allows the development of a convenient assay system for receptor binding that may be easily adapted for the screening of modulators (agonists and antagonists) of the interaction between an APP or an APLP and F-spondin or neurexin proteins. Modulators so identified may be useful for the treatment or prevention of Alzheimer's disease (AD).

### BACKGROUND OF THE INVENTION

Alzheimer's Disease and Amyloid-β Precursor Protein. Alzheimer's disease (AD) is a progressive neurodegenerative disorder that affects millions of people worldwide. AD is the leading cause for dementia in the elderly. See e.g. Selkoe, Trends Cell Biol. 1998;8:447-453; Ashe, Ann. N.Y. Acad. Sci. 2000;924:39-41; Masliah Ann. N.Y. Acad. Sci. 2000;924:68-75; Small et al., Nat. Rev. Neurosci. 2001;2:595-598; Chan et al., Neuromolecular Med. 2002;2:167-196; Selkoe, Science 2002;298:789-791. The primary clinical manifestation of AD is a discrete cognitive impairment in learning and memory. Sensory and motor functions remain relatively intact. The progressive memory loss in AD eventually results in the complete incapacitation of the patient.

Pathologically, the disease is characterized by lesions comprising neurofibrillary tangles, cerebrovascular amyloid deposits, and neuritic plaques. The cerebrovascular amyloid deposits and neuritic plaques contain amyloid-β peptide (Aβ). Aβ comprises a series of peptides that differ slightly in their N- and C-termini and are the physiological cleavage products of APP (see below). The major species of Aβ, Aβ40 and Aβ42, share a common N-terminus but extend C-terminally for 40 and 42 residues, respectively.

Studies performed in human patients, induced mouse mutants, and in vitro have provided overwhelming evidence that the primary and immediate cause of AD is overproduction of Aβ42, with or without Aβ40. See e.g. Price et al., Annu. Rev. Genet. 1998;32:461-493; Selkoe, Trends Cell Biol. 1998;8:447-453; Ashe, Ann. N.Y. Acad. Sci. 2000;924:39-41; Coulson et al., Neurochem. Int. 2000;36:175-184; Masliah Ann. N.Y. Acad. Sci. 2000;924:68-75; Small et al., Nat. Rev. Neurosci. 2001;2:595-598; Chan et al., Neuromolecular Med. 2002;2:167-196; Selkoe, Science 2002;298:789-791. Aβ42 is thought to form toxic aggregates or protofilaments that impair synapse function, decrease neuronal survival, and induce vascular amyloidosis, of which synapse damage may be the primary pathogenic event. Selkoe, Science 2002;298:789-791.

At present, it is believed that two parameters govern the probability with which an individual develops AD: 1) The rate of Aβ production, which is likely regulated and is probably linked to the function of APP; 2) The aggregation rate and toxicity of Aβ protofibrils, which may depend on the local ability of neurons to clear aggregates and on the vulnerability of these neurons to the aggregates. While enormous progress has been made in the understanding of APP cleavage by α-, β- and γ-secretases and hence the basic mode of production of Aβ through the analysis of mouse models for AD, the physiological functions of APP, the nature of the Aβ-induced synaptic pathology in AD, and the relation between APP function and AD all remain incompletely understood. Because the rate of Aβ formation and thus the occurrence of AD is likely a consequence of the normal physiological functions of APP, elucidation of these functions may lead to the development of new treatment modalities for AD.

Structure and Cleavage of APP and Its Homologs. Aβ is derived from amyloid-β precursor protein (APP). APP is a type I membrane protein that resembles a cell-surface receptor. APP is expressed in three major splice variants, referred to as APP₆₉₅, APP₇₅₁, and APP₇₇₀ based on the number of residues in human APP. All APP splice variants contain a large extracellular sequence, a single C-terminal transmembrane region (TMR), and short intracellular tail (FIG. 1). While its precise biological function has not yet been fully elucidated, many functions have been proposed for APP, including roles in axonal transport, neurite outgrowth, neuronal survival, transcriptional signaling, and synapse formation (see below).

The large extracellular region of APP contains four principal domains: a cysteine-rich N-terminal domain, an acidic sequence, an alternatively spliced Kunitz-type protease inhibitor domain, and a large central region referred to as CER for central extracellular region. These domains are separated from the TMR by a non-conserved linker sequence that contains the cleavage sites for α- and β-secretases (see below). The three principal splice variants differ primarily in the presence or absence of the Kunitz domain. Neurons mostly express APP₆₉₅, which lacks this domain. Palmert et al., Science 1988;241:1080-1084. Apart from the alternatively spliced Kunitz domain, little is known about the extracellular domains of APP. A crystal structure of the N-terminal cysteine-rich domain revealed a compact, disulfide bonded globular domain without significant similarities to other proteins. Rossjohn et al., Nat. Struct. Biol. 1999;6:327-331. The acidic sequence binds zinc with high affinity, but its function is unknown, as is that of the CER which is the largest conserved APP sequence. Bush et al., J. Biol. Chem. 1993;268:16109-16112; Bush et al., J. Biol. Chem. 1994;269:26618-26621.

APP is cleaved physiologically by site-specific proteases called α-, β-, and γ-secretases. Initially, α- and β-secretases cleave APP at defined extracellular sequences just outside of the TMR to release a large N-terminal extracellular fragment, called sAPP. Thereafter, γ-secretase cuts APP in the middle of the TMR to generate small extracellular peptides, the Aβ peptides, and a C-terminal fragment comprising half of the TMR and the full cytoplasmic tail. See e.g. Price et al., Ann. Rev. Genet. 1998;32:461-493; Selkoe, Trends Cell Biol. 1998;8:447-453; Bayer et al., Mol. Psychiatry 1999;4:524; Haass et al., Science 1999;286:916-919. The Aβ peptides produced by the γ-secretase-mediated cleavage of APP include Aβ340 and Aβ342, which are thought to be the major pathogenic agents in AD. The small intracellular fragment produced by this same cleavage reaction, called APP intracellular domain (AICD), has recently been shown to act as an intracellular signaling molecule that regulates gene transcription. See Cao and Südhof, Science 2001;293:115-120.

Two proteins that are closely related to APP, called APLP1 and APLP2, are expressed in mammals. See Sprecher et al., Biochemistry 1993;32:4481-4486; Wasco et al., Proc. Natl. Acad. Sci. U.S.A. 1993;89:10758-10762; Wasco et al., Nat. Genet. 1993;5:95-100; Sandbrink et al., Biochim. Biophys. Acta 1994;1219:167-170; Slunt et al., J. Biol. Chem. 1994;269:2637-2644. Both APLPs exhibit a similar domain structure as APP (except that APLP1 lacks a Kunitz inhibitor domain), and are cleaved at least by α- and γ-secretases, also leading to the secretion of a large ectodomain (sAPLP). Slunt et al., J. Biol. Chem. 1994;269:2637-2644; Lorent et al., Neuroscience 1995;65:1009-1025. The similarity between APP and the APLPs indicates that they may be functionally redundant. This has been confirmed in knock-out (KO) mouse studies showing that single APP, APLP1, or APLP2 KO mice are viable, but double KOs of APP with either APLP1 or APLP2 are lethal. von Koch et al., Neurobiol. Aging 1997;18:661-669; Heber et al., J. Neurosci. 2000;20:7951-7963.

APP binding proteins. A large number of proteins have been described that bind to the AICD, including G₀ (Nishimoto et al., Nature 1993;362:75-79; Brouillet et al., J. Neurosci. 1999;19:1717-1727), Fe65 (Fiore et al., J. Biol. Chem. 1995;270:30853-30856; McLoughlin and Miller, FEBS Lett. 1996;397:197-200; Borg et al., Mol Cell Biol 1996;16:6229-6241), Mints/X11s (McLoughlin and Miller, FEBS Lett. 1996;397:197-200; Borg et al., Mol Cell Biol 1996;16:6229-6241; Biederer et al., J. Neurosci. 2002;22:7340-7351), APP-BP1 (Chow et al., J. Biol. Chem. 1996;271:11339-11346), Pat1 (Zheng et al., Proc. Natl. Acad. Sci. U.S.A. 1998;95:14745-14750), disabled-1 (Trommsdorff et al., J. Biol. Chem. 1998;273:33556-33560; Homayouni et al., J. Neurosci. 1999;19:7507-7515; Howell et al., Mol. Cell Biol. 1999;19:5179-5188), kinesin light chain (Kamal et al., Nature 2001;414:643-648), JIP-1b (Matsuda et al., J. Neurosci. 2001;21:6597-6607), and Shc (Tarr et al., J. Biol. Chem. 2002;277:16798-16804).

Most of these proteins exhibit interesting domain structures and have properties consistent with a role in signal transduction via APP. For example, Mints/X11 and Fe65 are multidomain proteins of unknown function that are primarily expressed in brain. Both contain a PTB domain that binds to the NPTY sequence in the AICD, although their binding specificity differs. Fiore et al., J. Biol. Chem. 1995;270:30853-30856; McLoughlin and Miller, FEBS Lett. 1996;397:197-200; Borg et al., Mol Cell Biol 1996;16:6229-6241; Biederer et al., J. Neurosci. 2002;22:7340-7351. Mints/X11s are composed of a unique N-terminal sequence followed by the APP-binding PTB domain and two C-terminal PDZ-domains. Okamoto and Südhof, J. Biol. Chem. 1997;272:31459-31464; Okamoto and Südhof, J. Cell Biol. 1998;77:161-165. Fe65 also has a unique N-terminal sequence that, however, is followed by a central WW domain and two C-terminal PTB-domains, the second of which binds to APP.

Co-transfection of Fe65 and Mints with APP results in small changes of Aβ production. Biederer et al., J. Neurosci. 2002;22:7340-7351,67-70. In addition, Mints stabilize APP levels. Biederer et al., J. Neurosci. 2002;22:7340-7351,69. Fe65 is a potent transactivator of transcription when bound to the AICD (Cao and Südhof, Science 2001;293:115-120) or the analogous fragment of APLPs (Scheinfeld et al., J. Biol. Chem. 2002;277:44195-44201), whereas Mints interfere with APP-dependent transcriptional activation (Biederer et al., J. Neurosci. 2002;22:7340-7351). Mints may function at the synapse because they bind to Munc18-1, which is essential for synaptic vesicle exocytosis, and to Ca²⁺-channels (Okamoto and Südhof, J. Biol. Chem. 1997;272:31459-31464; Maximov et al., J. Biol. Chem. 1999;274:24453-24456), and Munc18 potentiates the stabilization of APP by Mint 1 (Ho et al., J. Biol. Chem. 2002;277:27021-27028).

In contrast to the wealth of knowledge regarding proteins that bind to the AICD of APP, little is known about proteins that bind to the extracellular domains of neuronal APP. However, because proteins that bind to APP may exert small but, over time, profound effects on the levels of APP in the cell and on the production of Aβ, as shown above for proteins that bind the AICD, thereby influencing the development of AD, the identification of ligands that bind to the extracellular domain of APP may be critical in the creation of novel treatments for this neurodegenerative disease.

Functions of APP. Multiple functions have been proposed for APP, mainly based on in vitro experiments. See e.g. Mattson, Physiol. Rev. 1997;77:1081-1132; Koo, Traffic 2002;3:763-770. Some of these functions—such as effects mediated by the alternatively spliced Kunitz domain—apply only to a subset of APP variants. Other functions (e.g. a role for APP in neuronal survival or neurite extension) have been made unlikely by the results of the KO experiments. Heber et al., J. Neurosci. 2000;20:7951-7963. Double or triple KO mice of APP/APLPs die in the first postnatal week because of a failure to feed, but do not exhibit structural or morphological changes in brain, suggesting that APP and APLPs are not essential for axonal outgrowth, neurite extension, neuronal survival, or synapse formation. Heber et al., J. Neurosci. 2000;20:7951-7963.

Four major ideas about the function of APP are currently proposed. First, a role for APP in axonal transport of a subset of vesicles. Kamal et al., Neuron 2000;28:449-459; Gunawardena et al., Neuron 2001;32:389-401; Koo, Traffic 2002;3:763-770. This role is based on the prominent axonal transport of APP and APLPs, the direct binding of the cytoplasmic tail of APP to kinesin light chain, and the changes in axon morphology and axonal transport observed in Drosophila mutants of the APP homolog. Torroja et al., Curr. Biol. 1999;9:489-492; Gunawardena et al., Neuron 2001;32:389-401. This attractive idea would agree well with the predominant localization of APP to the trans-Golgi (the starting point of transported vesicles), the synaptic phenotype of AD, and the secretion of Aβ from nerve terminals. However, it is somewhat puzzling that the APP/APLP double and triple KO mice do not exhibit a major morphological phenotype as would be expected from this hypothesis. Heber et al., J. Neurosci. 2000;20:7951-7963.

A second proposed function of APP is in intracellular signaling via kinases or the cytoskeleton. This idea is based on the multiple interactions of APP with cytoplasmic signaling molecules. Nishimoto et al., Nature 1993;362:75-79; Fiore et al., J. Biol. Chem. 1995;270:30853-30856; Borg et al., Mol Cell Biol 1996;16:6229-6241; Chow et al., J. Biol. Chem. 1996;271:11339-11346; McLoughlin and Miller, FEBS Lett. 1996;397:197-200; Trommsdorff et al., J. Biol. Chem. 1998;273:33556-33560; Zheng et al., Proc. Natl. Acad. Sci. U.S.A. 1998;95:14745-14750; Brouillet et al., J. Neurosci. 1999;19:1717-1727; Homayouni et al., J. Neurosci. 1999;19:7507-7515; Howell et al., Mol. Cell Biol. 1999;19:5179-5188; Kamal et al., Nature 2001;414:643-648; Matsuda et al., J. Neurosci. 2001;21:6597-6607; Tarr et al., J. Biol. Chem. 2002;277:16798-16804. However, no signal transduction event that is directly dependent on APP has been observed.

A third role has been proposed for APP in regulating transcription, based on the potent transactivation of target genes by the AICD/Fe65. Cao and Südhof, Science 2001;293:115-120; Scheinfeld et al., J. Biol. Chem. 2002;277:44195-44201. However, only one potential target gene has been identified so far. Baek et al., Cell 2002;110:55-67.

APP also has been implicated in the formation and maintenance of synapses, based on the interaction of APP with Mints/X11 which in turn bind to Munc18-1 (Okamoto and Südhof, J. Biol. Chem. 1997;272:31459-31464), and on the strong effects of APP overproduction on synapse formation in Drosophila neuromuscular junctions (Torroja et al., J. Neurosci. 1999;19:7793-7803). However, the latter effects could also have been indirect, and the physiological role of Mints and their binding to APP is unclear.

AD as a synaptic disease. The fact that in all familial forms of AD, Aβ metabolism is altered in a way that fosters Aβ42 production, aggregation, or deposition suggests that Aβ42 is the pathogenic agent in AD. See e.g. Price et al., Annu. Rev. Genet. 1998;32:461-493; Selkoe, Trends Cell Biol. 1998;8:447-453; Ashe, Ann. N.Y. Acad. Sci. 2000;924:39-41; Coulson et al., Neurochem. Int. 2000;36:175-184; Masliah Ann. N.Y. Acad. Sci. 2000;924:68-75; Small et al., Nat. Rev. Neurosci. 2001;2:595-598; Chan et al., Neuromolecular Med. 2002;2:167-196; Selkoe, Science 2002;298:789-791.

Significant evidence suggests that Aβ42 causes AD by damaging synapses. Selkoe, Science 2002;298:789-791. For example, the fast axonal transport of APP to nerve terminals and APP cleavage in the terminals places Aβ at the synapse. Koo et al., Proc. Natl. Acad. Sci. U.S.A. 1990;87:1561-1565; Morin et al., J. Neurochem. 1993;61:464-473; Amaratunga and Fine, J. Biol. Chem. 1995;270:17268-17172; Thinakaran et al., J. Neurosci. 1995;15:6314-6326; Tienari et al., EMBO J. 1996;15:5218-5229; Buxbaum et al., J. Neurosci. 1998;18:9629-9637; Lyckman et al. J. Biol. Chem. 1998;273:11100-11106; Lazarov et al., J. Neurosci. 2002;22:9785-9793; Sheng et al., J. Neurosci. 2002;22:9794-9799. Furthermore, the symptoms of AD, especially loss of cognitive functions, best correlate with synapse loss. See e.g. DeKosky and Scheff, Ann. Neurol. 1990;27:457-464; Scheff et al., Neurobiol Aging. 1990;11:29-37).

In transgenic mice overexpressing Aβ42, the major behavioral and electrophysiological impairments correlate with synapse loss, not with amyloid plaque formation. See e.g. Irizarry et al., J. Neuropathol. Exp. Neurol. 1997;56:965-973; Hsia et al., Proc. Natl. Acad. Sci. U.S.A. 1999;96:3228-3233; Dodart et al., Neurobiol Dis. 2000;7:71-85; Buttini et al., J. Neurosci. 2002;22:10539-48; Kotilinek et al., J. Neurosci. 2002;22:6331-6335; Westerman et al., J. Neurosci. 2002;22:1858-1867.).

Lewy bodies are observed in ˜60% of AD cases (Kazee and Han, Arch. Pathol. Lab. Med. 1995;119:448; Lippa et al., Am. J. Pathol. 1998;153:1365-1370-453. 88,89). Since Lewy bodies are primarily composed of a presynaptic protein called α-synuclein that is involved in Parkinson's disease (Lotharius and Brundin, Nat. Rev. Neurosci. 2002;3:932-942), Aβ toxicity may induce presynaptic α-synuclein aggregation in a subset of cases.

F-spondin. F-spondin is a secreted multi-domain protein that promotes neural cell adhesion and neurite extension. Feinstein et al., Development 1999;126:3637-3648. This protein is composed of an N-terminal 200 residue region that is homologous to reelin, a central “spondin” domain and six C-terminal thrombospondin type 1 repeats (residues 440-807). Feinstein et al., Development 1999;126:3637-3648.

F-spondin is expressed at high levels in the floor plate of the developing spinal cord (Klar et al. Cell 1992;69:95-110). However, F-spondin is also ubiquitously present in embryonic and adult tissues (Miyamoto et al., Arch. Biochem. Biophys. 2001;390:93-100), and axotomy of adult sciatic nerve causes massive upregulation of F-spondin (Burstyn-Cohen et al., J. Neurosci. 1998; 18:8875-8885). Recombinant F-spondin promotes neural cell adhesion and neurite extension, suggesting that it may function to stimulate axonal extension and repair. Klar et al. Cell 1992;69:95-110; Burstyn-Cohen et al., J. Neurosci. 1998;18:8875-8885. F-spondin also has been implicated in axonal pathfinding, cell-cell interactions, and neural regeneration. See e.g. Klar et al., 1992, Cell 69:95-110; Burstyn-Cohen et al., 1998, J. Neurosci. 18:8875-8885; Burstyn-Cohen et al., 1999, Neuron 23:233-246; Debby-Brafman et al., 1999, Neuron 22:475-488; Miyamoto et al., Arch. Biochem. Biophys. 390:93-110; Terai et al., 2001, J. Cell Physiol. 188:394-402; Tzarfati et al., 2001, Proc. Natl. Acad. Sci. USA 98:4722-4727. F-spondin binds to the cell surface of neurons, but no neuronal receptor has been identified.

Recombinant F-spondin stimulates proliferation of vascular smooth muscle cells, suggesting that, consistent with its ubiquitous expression, F-spondin also acts on non-neuronal cells. Miyamoto et al., Arch. Biochem. Biophys. 2001;390:93-100. Thus, F-spondin likely mediates cellular responses in brain and periphery by binding to specific cell-surface receptors.

The Neurexin Family of Proteins. Neurexins are neuron-specific cell-surface proteins that are thought to function at the synapse. Ushkaryov et al., Science 1992;257:50-56. In mammals, three genes each encode an α- and a β-neurexin that are transcribed from separate promoters, and are diversified by extensive alternative splicing. Missler and Südhof, Trends Genet. 1998;14:20-25; Tabuchi and Südhof, Genomics 2002;79:849-859. Neurexins interact with neuroligins and dystroglycan, which in turn may act as postsynaptic cell adhesion molecules by binding to presynaptic neurexins (Ichtchenko et al., Cell 1995;81:435-443; Nguyen and Südhof, J. Biol. Chem. 1997;272:26032-26039; Scheiffele et al., Cell 2000;101:657-669; Sugita et al., J. Cell Biol. 2001; 154:435-445; Moore et al., Nature 2002;418:422-425), and with neurexophilins which resemble hormone-like proteins (Missler and Südhof, J. Neurosci. 1998;18:3630-3638). KOs of α-neurexins cause a severe synaptic phenotype.

In accordance with the present invention, F-spondin and neurexin proteins have been identified as endogenous ligands for APP. The identification of a specific interaction between APP and F-spondin and neurexin proteins, respectively, permits the development of various receptor-binding assays to identify modulators (agonists and antagonists) of the F-spondin/APP and neurexin/APP binding reactions. Such modulators may be useful for the treatment or prevention of Alzheimer's disease (AD).

### SUMMARY OF THE INVENTION

The present invention provides the discovery that F-spondin and the neurexin family of proteins are endogenous ligands of the amyloid-β precursor protein (APP) or APP-like proteins (APLPs). Thus, in one aspect, the invention provides a composition comprising an isolated F-spondin polypeptide specifically bound to an APP or APLP polypeptide. In another aspect, the invention provides a composition comprising an isolated neurexin polypeptide specifically bound to an APP or APLP polypeptide. In specific embodiments, the F-spondin or neurexin polypeptides are detectably labeled. These compositions can be employed in a screen for compounds that modulate the binding of an APP or an APLP by either F-spondin or neurexin polypeptides.

The present invention further provides a method of screening for modulators of the binding of an APP or an APLP by F-spondin or neurexin proteins. In one embodiment, this method comprises detecting a change in binding activity of a detectably-labeled F-spondin or neurexin polypeptide to an APP or an APLP in the presence or absence of a candidate compound under conditions that permit binding of the F-spondin or neurexin polypeptide to an APP or an APLP, wherein detection of a change in binding activity indicates that the candidate compound is a modulator of the binding of an APP or an APLP by F-spondin or a neurexin protein. Because binding of F-spondin to APP dramatically reduced the cleavage of APP by α-, β-, and γ-secretases and hence the production of amyloidogenic APP cleavage products (i.e. Aβ40 and Aβ42), modulators of the binding of an APP or an APLP by F-spondin may be useful in the treatment or prevention of AD.

These and other aspects of the invention are described in greater detail in the Detailed Description and Examples, infra.

### BRIEF DESCRIPTION OF THE DRAWINGS

FIGS. 1A-1B. Binding of F-spondin to immobilized APP. A. Domain structure of APP (top) and diagram of various APP vectors employed for the present study (bottom). B. Affinity chromatography of secreted myc-tagged recombinant F-spondin on immobilized APP proteins.

FIGS. 2A-2E. Binding of APP to immobilized F-spondin. A. Domain structure of F-spondin (top) and parts of F-spondin included in the various Ig-fusion vectors employed for the present study (bottom). The positions of the two N-glycosylation sites are indicated. B. Pulldown of full-length APP695. C. Pulldown of APP deletion mutants (see panel A in FIG. 1 for extent of the deletions) with full-length Ig-F spondin. D. Comparison of the ability of immobilized full-length F-spondin to affinity-purify APP, APLP1, and APLP2 expressed in transfected COS cells, and visualized with antibodies to the C-termini of indicated proteins. E. Nucleotide and amino acid sequences of the F-spondin Ig-fusion proteins Ig-F-spondin.1, Ig-F-spondin.2, Ig-F-spondin.3, Ig-F-spondin.4, Ig-F-spondin.5, and Ig-F-spondin.6 depicted schematically in panel A.

FIGS. 3A-3C. Lack of an interaction of APP with Mindin. A. Domain structure of Mindin (SP=signal peptide; a spondin-like domain and, TSR=thrombospondin repeat). B. Pulldown of myc-tagged Mindin with immobilized GST-CAPPD fusion protein. C. Pulldown of APP with a Ig-Mindin fusion protein.

FIGS. 4A-4B. F-spondin inhibits cleavage of APP by BACE 1. A. Immunoblot of HEK293 cells that were transfected without or with BACE 1, Ig-C, or Ig-F spondin as indicated. Numbers on the left indicate positions of molecular weight markers. B. Quantification of the results shown in panel A.

FIGS. 5A-5B. Titration of F-spondin mediated inhibition of APP cleavage by BACE 1. A. Relative levels of proteins expressed in an experiment similar to that described in FIG. 4. B. Ratio of CTF to full-length APP as a function of increasing amount of F-spondin.

FIGS. 6A-6C. Effect of F-spondin on APP-dependent transactivation of Gal4-Tip60-mediated transcription. A. F-spondin inhibits APP-dependent transactivation. B. Comparison of the effects of multiple Ig-fusion proteins on the APP-dependent transactivation of Gal4-Tip60. C. Increasing concentrations of APP are unable to rescue the F-spondin dependent inhibition of APP-dependent transactivation of Gal4-Tip60.

### DETAILED DESCRIPTION OF THE INVENTION

The present invention is based, in part, on the discovery that the amyloid-β precursor protein (APP), a molecule previously known to be involved in the pathophysiology of Alzheimer's disease (AD), serves as a cellular receptor for the endogenous ligands F-spondin and proteins of the neurexin family. This discovery indicates that APP activity, the rate of formation of the amyloidogenic peptide amyloid-β (Aβ), and hence the pathophysiological status of AD, may be influenced by compounds that modulate or mimic the specific interactions between an APP and F-spondin or neurexins.

Because APP and APLP possess highly homologous extracellular domain structures, the present invention also demonstrates that APLPs serve as cellular receptors for the endogenous ligands F-spondin and proteins of the neurexin family.

The present invention therefore provides a binding assay for modulators of the interaction between an APP or an APLP and F-spondin or neurexin proteins. Said binding assay could be employed as a means of identifying compounds that promote, block, or otherwise modulate these associations. The compounds so identified could be used to further elucidate the function of APP or APLPs, or as therapeutic agents to prevent or alleviate AD, to prevent synaptic degeneration, and to enhance cognitive functions and memory.

Although the various components involved in the present invention, such as the various forms of APP, APLP, F-spondin and the neurexin proteins, are well known, their ability to directly interact was not heretofore known. Thus, other aspects of the invention provide novel compositions comprising an APP₆₉₅ polypeptide, an APLP1 polypeptide, or an APLP2 polypeptide specifically bound to an isolated F-spondin polypeptide, an isolated α-neurexin polypeptide or an isolated β-neurexin polypeptide. These compositions may be used to determine the specificity and affinity of binding of other ligands to APP or APLP, or for the identification of agents that modulate these processes. Such compositions preferably are prepared in an isotonic, buffered aqueous solution.

### Definitions—General

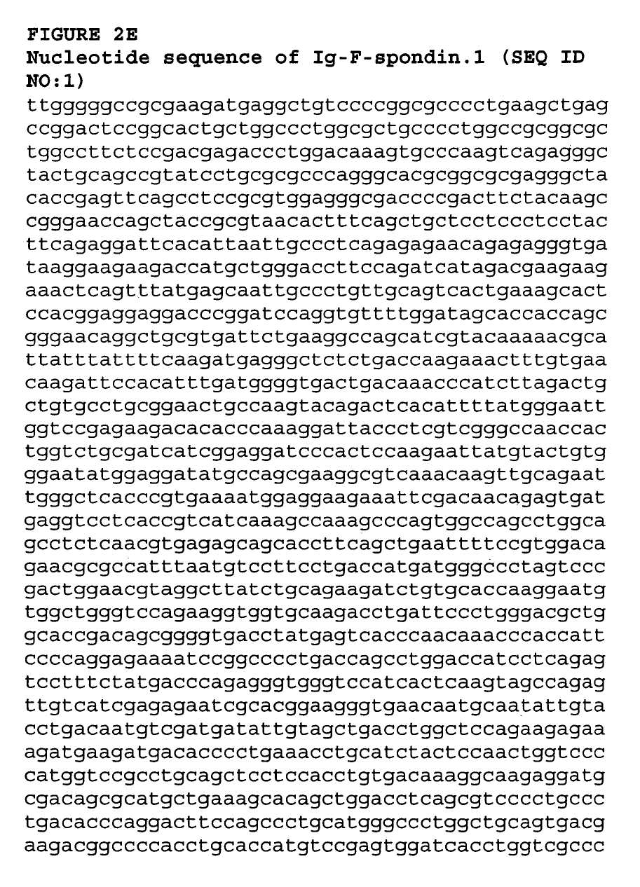

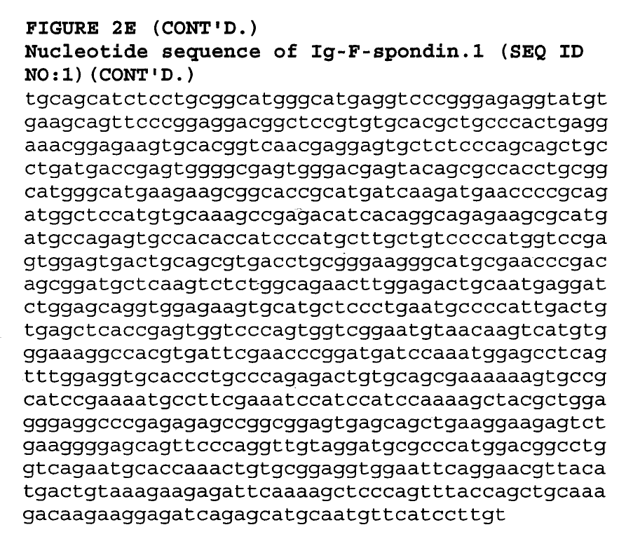

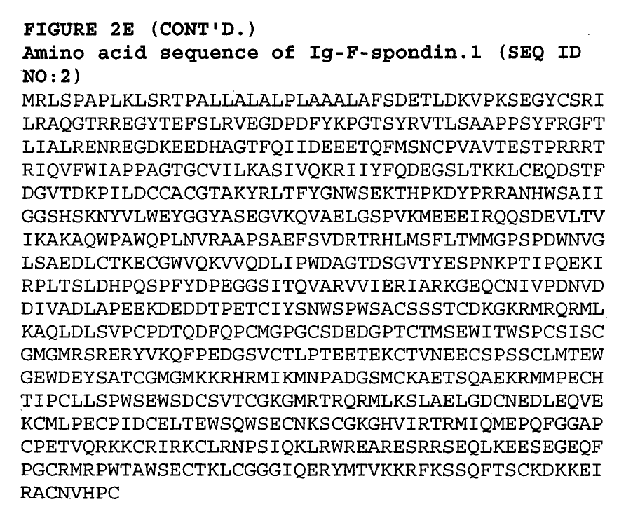

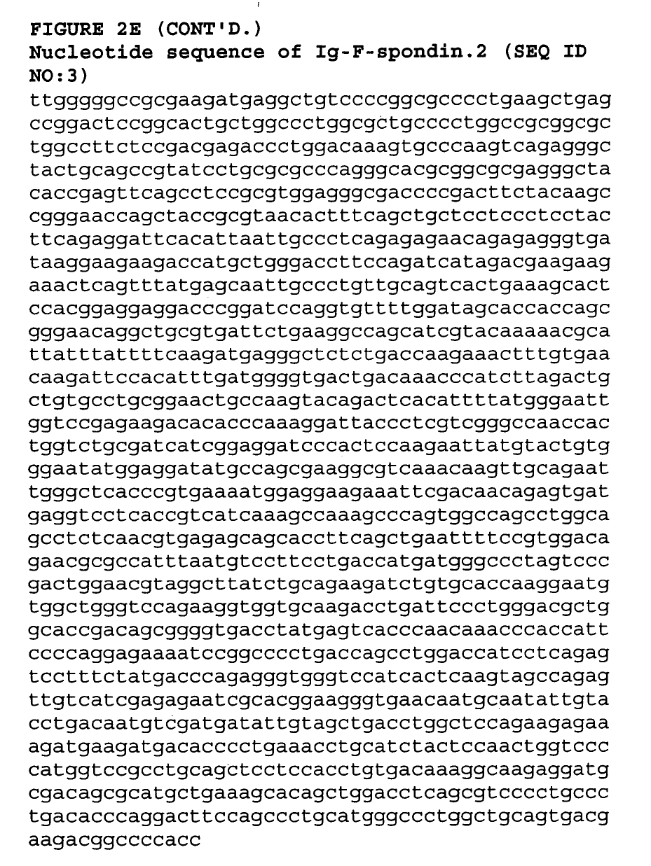

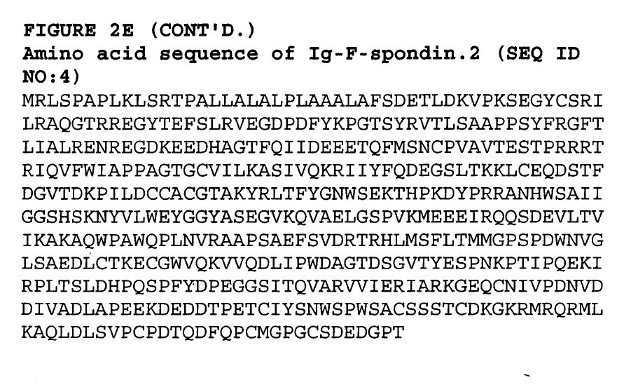

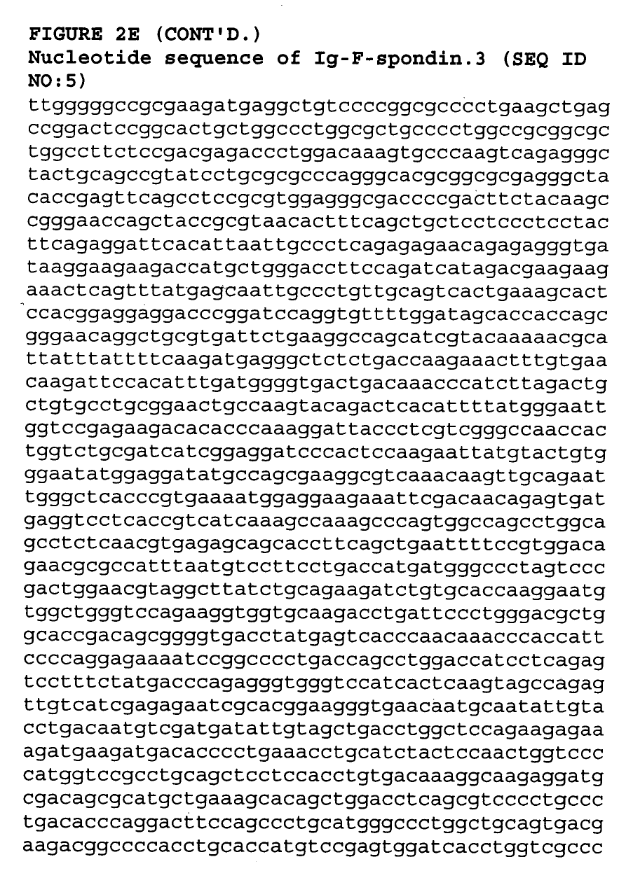

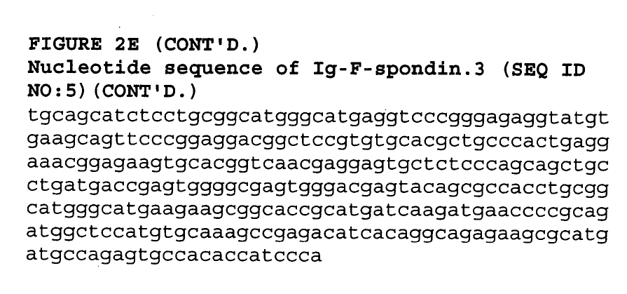

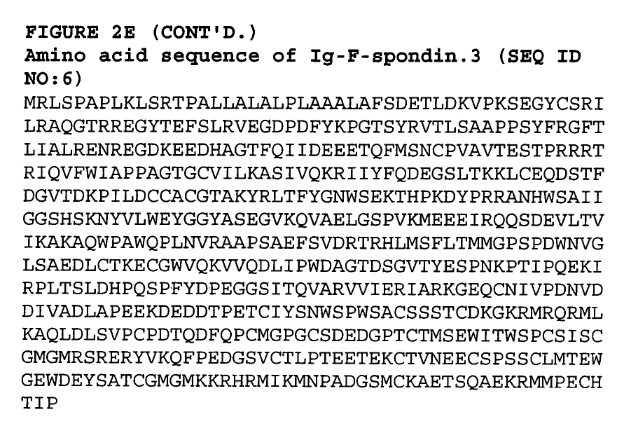

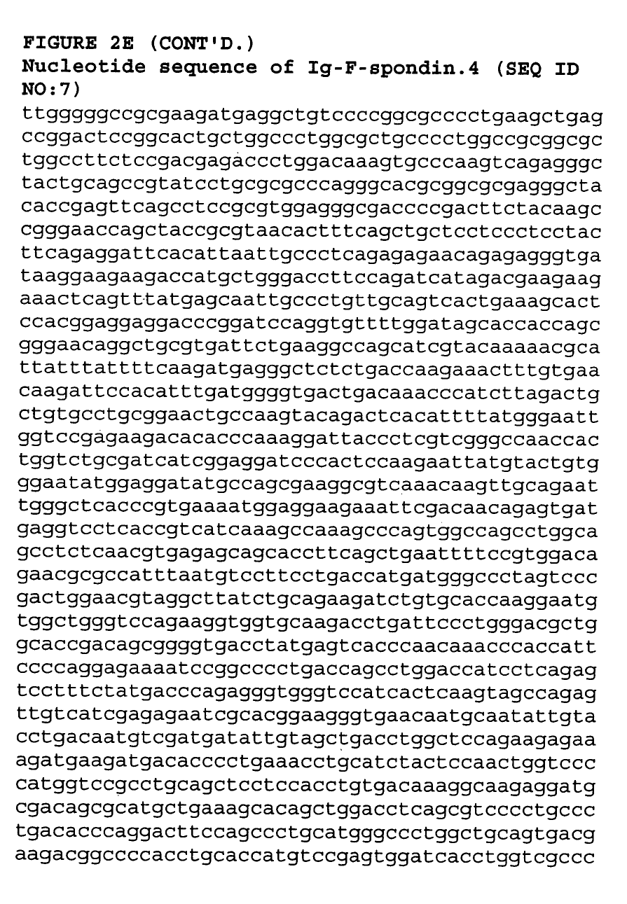

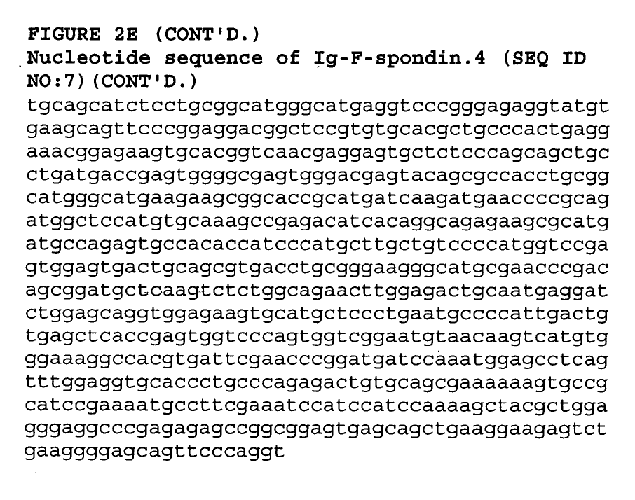

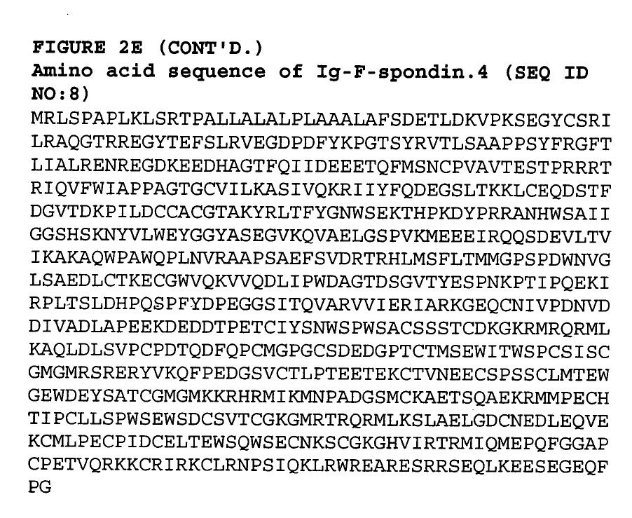

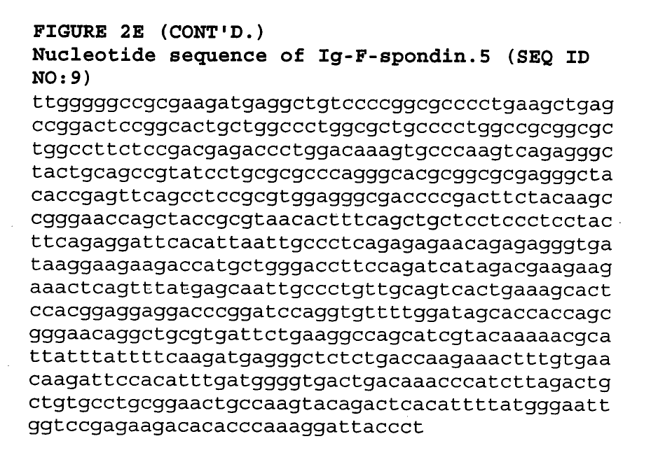

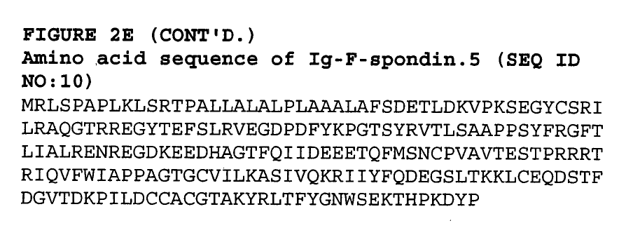

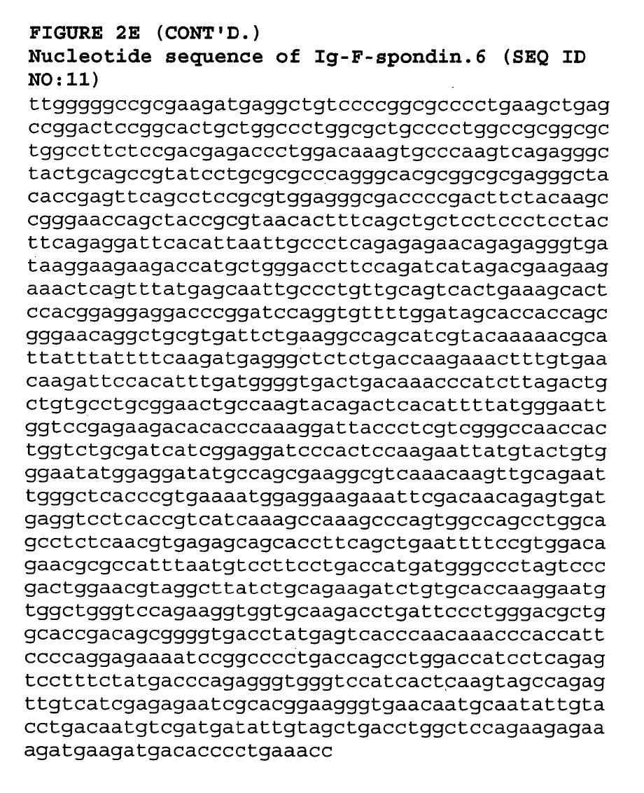

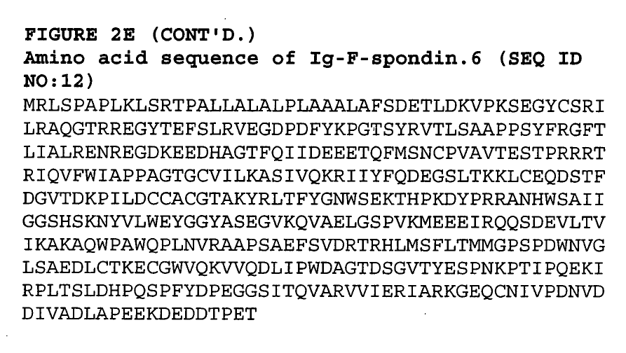

As used herein, an “F-spondin polypeptide” means the full-length F-spondin protein, an F-spondin fusion protein, or a fragment of the F-spondin protein that can bind to APP or its homologs and that can modulate APP-mediated signaling. Preferred embodiments of F-spondin polypeptides are depicted in FIG. 2A, and their sequences are shown in FIG. 2E. Particularly preferred embodiments are those subfragments of F-spondin that comprise the spondin domain.

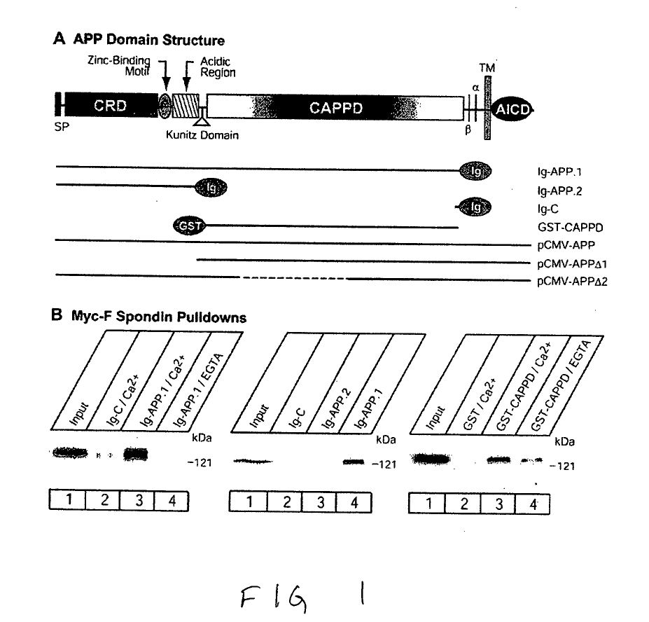

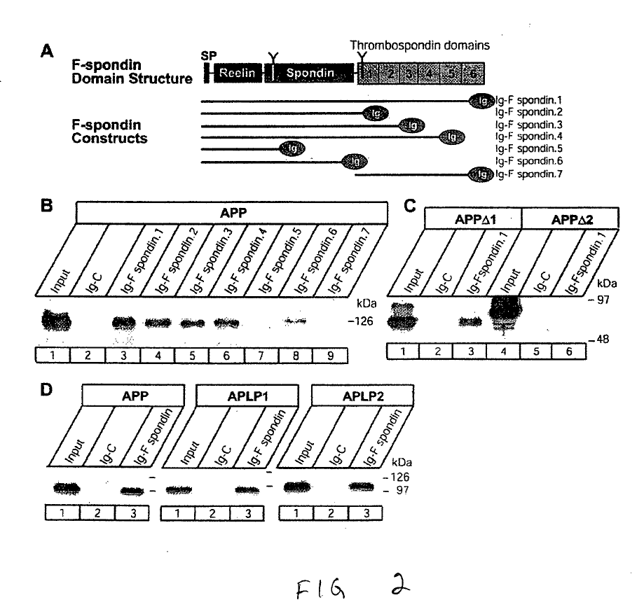

As used herein, an “APP polypeptide” refers to APP family members that are characterized by (i) structural similarity as depicted schematically in FIG. 1A; (ii) cleavage by α-, β-, and γ-secretases, and (iii) binding to Fe65, Tip60 and/or Mints/X11. Preferably the APP polypeptide binds to F-spondin or to a member of the neurexin family of proteins. In a specific, preferred embodiment, the APP is APP₆₉₅. Other non-limiting examples of members of this group presently include APP₇₅₁, APP₇₇₀, and the APP-like proteins APLP1 and APLP2.

“Detectably labeled” means that a polypeptide or other binding partner of a binding pair (including, for example, a small molecule agonist or antagonist of F-spondin discovered in a screen of the invention) comprises a molecular entity that directly provides a signal or that interacts with a secondary molecule that is itself detectably labeled. An example of the former is a reporter protein, such as alkaline phosphatase, luciferase, green fluorescent protein, or horseradish peroxidase. An example of the latter is biotin (which binds avidin or streptavidin), an epitope tag, or a hapten group (each of which bind specific antibodies). Any of the labels described herein can be used to detect binding of the secondary binding molecule. In addition to reporter proteins, other labels for direct signal detection include colloidal gold, colored latex beads, magnetic beads, fluorescent labels (e.g. fluorescene isothiocyanate (FITC), phycoerythrin (PE), Texas red (TR), rhodamine, free or chelated lanthanide series salts, especially Eu³⁺, to name a few fluorophores), chemiluminescent molecules, radio-isotopes (¹²⁵I, ³²P, ³⁵S, chelated Tc, etc.), or magnetic resonance imaging labels.

The term “signal transduction pathway” as used in this invention refers to the intracellular mechanism by which APP induces an alteration of cell function or activity, e.g. transcriptional activation, synaptic function, or neuronal survival or function. Key features of the signal transduction pathway dissected herein is the association of an APP with F-spondin or a neurexin peptide, cleavage of the APP by α-, β-, and/or γ-secretases, and generation of sAPP, Aβ and APP intracellular domain (AICD) peptides.

The term “element of a signal transduction pathway” refers to a signal transduction factor that is activated as a result of cleavage of an APP, particularly APP₆₉₅. In accordance with the present invention, elements of the APP signal transduction pathway include F-spondin or the neurexin family of proteins, an APP or homologous proteins, Fe65, Tip60, Mints/X11 and the α-, β-, and/or γ-secretases. A “signal” in such a pathway can refer to binding of F-spondin or a neurexin peptide to an APP, cleavage of an APP, or activation of additional elements or factors in the pathway. For example, the formation of a tripartite complex between AICD, Fe65, and Tip60 leads to activation of gene transcription, and this may constitute the signal. Cao and Südhof, Science 2001;293:115-120.

“APP-mediated signaling” and “APP-mediated signal transduction” refer to the cascade of cellular events that result from binding of F-spondin or the neurexin proteins to APP or its homologs or from the cleavage of an APP or an APLP in a cell that expresses an APP or an APLP, particularly APP₆₉₅.

Cells for use in accordance with the invention express a functional APP or APLP molecule, e.g. APP₆₉₅. Cells that express APP₆₉₅ endogenously include but are not limited to neuronal cells. Alternatively, as mentioned above, cells expressing an APP or an APLP can be generated using recombinant technology, preferably in conjunction with Fe65, Tip60 and/or Mints/X11.

The term “inhibitor” is used herein to refer to a compound that can block signaling in the signal transduction pathway described herein. Such an inhibitor may block the pathway at any point, from blocking binding of F-spondin or a neurexin polypeptide to an APP or an APLP, to blocking function of intracellular signal pathways induced by F-spondin binding to an APP or an APLP or by cleavage of an APP or an APLP.

The term “agonist” is used herein to refer to a compound that can induce signaling in the F-spondin/APP, F-spondin/APLP, neurexin/APP, or neurexin/APLP signal transduction pathways described herein. Such an agonist may induce the pathway at any point. Preferably an agonist discovered in accordance with the instant invention mimics the binding of F-spondin or neurexin to an APP.

The term “antagonist” is used herein to refer to a compound that can block signaling in the F-spondin/APP or neurexin/APP signal transduction pathways described herein. Such an antagonist may induce the pathway at any point. Preferably an antagonist discovered in accordance with the instant invention blocks the binding of F-spondin or neurexin to an APP.

“Screening” refers to a process of testing one or a plurality of compounds (including a library of compounds) for some activity. A “screen” is a test system for screening. Screens can be primary, i.e. an initial selection process, or secondary, e.g. to confirm that a compound selected in a primary screen (such as a binding assay) functions as desired (such as in a signal transduction assay). Screening permits the more rapid elimination of irrelevant or non-functional compounds, and thus selection of more relevant compounds for further testing and development. “High throughput screening” involves the automation and robotization of screening systems to rapidly screen a large number of compounds for a desired activity.

As used herein, the term “isolated” means that the referenced material is removed from the environment in which it is normally found. Thus, an isolated biological material can be free of cellular components, i.e. components of the cells in which the material is found or produced in nature. In the case of nucleic acid molecules, an isolated nucleic acid includes a PCR product, an isolated mRNA, a cDNA, or a restriction fragment. In another embodiment, an isolated nucleic acid is preferably excised from the chromosome in which it may be found, and more preferably is no longer joined to non-regulatory, non-coding regions, or to other genes, located upstream or downstream of the gene contained by the isolated nucleic acid molecule when found in the chromosome. In yet another embodiment, the isolated nucleic acid lacks one or more introns.

Isolated nucleic acid molecules include sequences inserted into plasmids, cosmids, artificial chromosomes, and the like. Thus, in a specific embodiment, a recombinant nucleic acid is an isolated nucleic acid. An isolated protein may be associated with other proteins or nucleic acids, or both, with which it associates in the cell, or with cellular membranes if it is a membrane-associated protein. As used herein, a membrane protein, such as APP₆₉₅, expressed in a heterologous host cell (i.e. a host cell genetically engineered to express the membrane protein) is regarded as “isolated.” An isolated organelle, cell, or tissue is removed from the anatomical site in which it is found in an organism. An isolated material may be, but need not be, purified.

The term “purified” as used herein refers to material that has been isolated under conditions that reduce or eliminate the presence of unrelated materials, i.e. contaminants, including native materials from which the material is obtained. For example, a purified protein is preferably substantially free of other proteins or nucleic acids with which it is associated in a cell; a purified nucleic acid molecule is preferably substantially free of proteins or other unrelated nucleic acid molecules with which it can be found within a cell. As used herein, the term “substantially free” is used operationally, in the context of analytical testing of the material. Preferably, purified material substantially free of contaminants is at least 50% pure; more preferably, at least 90% pure, and more preferably still at least 99% pure. Purity can be evaluated by chromatography, gel electrophoresis, immunoassay, composition analysis, biological assay, and other methods known in the art.

Methods for purification are well known in the art. For example, nucleic acids can be purified by precipitation, chromatography (including preparative solid phase chromatography, oligonucleotide hybridization, and triple helix chromatography), ultracentrifugation, and other means. Polypeptides and proteins can be purified by various methods including, without limitation, preparative disc-gel electrophoresis, isoelectric focusing, HPLC, reversed-phase HPLC, gel filtration, ion exchange and partition chromatography, precipitation and salting-out chromatography, extraction, and countercurrent distribution. For some purposes, it is preferable to produce the polypeptide in a recombinant system in which the protein contains an additional sequence tag that facilitates purification, such as, but not limited to, a polyhistidine sequence, or a sequence that specifically binds to an antibody, such as FLAG and GST. The polypeptide can then be purified from a crude lysate of the host cell by chromatography on an appropriate solid-phase matrix. Alternatively, antibodies produced against the protein or against peptides derived therefrom can be used as purification reagents. Cells can be purified by various techniques, including centrifugation, matrix separation (e.g. nylon wool separation), panning and other immunoselection techniques, depletion (e.g. complement depletion of contaminating cells), and cell sorting (e.g. fluorescence activated cell sorting (FACS)). Other purification methods are possible. A purified material may contain less than about 50%, preferably less than about 75%, and most preferably less than about 90%, of the cellular components with which it was originally associated. The “substantially pure” indicates the highest degree of purity that can be achieved using conventional purification techniques known in the art.

In a specific embodiment, the term “about” or “approximately” means within 20%, preferably within 10%, and more preferably within 5% of a given value or range. Alternatively, particularly in biology, the term “about” can mean within an order of magnitude of a given value, and preferably within one-half an order of magnitude of the value.

Thus, in accordance with the present invention there may be employed conventional molecular biology, microbiology, and recombinant DNA techniques within the skill of the art. Such techniques are explained fully in the literature. See e.g. Sambrook, Fritsch & Maniatis, Molecular Cloning: A Laboratory Manual, Second Edition (1989) Cold Spring Harbor Laboratory Press, Cold Spring Harbor, N.Y. (hereinafter “Sambrook et al., 1989”); DNA Cloning: A Practical Approach, Volumes I and II (D. N. Glover ed. 1985); Oligonucleolide Synthesis (M. J. Gait ed. 1984); Nucleic Acid Hybridization (B. D. Hames & S. J. Higgins eds. 1985); Transcription And Translation (B. D. Hames & S. J. Higgins eds. 1984); Animal Cell Culture (R. I. Freshney, ed. 1986); Immobilized Cells And Enzymes (IRL Press, 1986); B. Perbal, A Practical Guide To Molecular Cloning, 1984; F. M. Ausubel et al. eds., Current Protocols in Molecular Biology, John Wiley & Sons, Inc., 1994.

### Definitions—Molecular Biology

A “nucleic acid molecule” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”). A “recombinant DNA molecule” is a DNA molecule that has undergone a molecular biological manipulation.

The term “host cell” means any cell of any organism that is selected, modified, transformed, grown, or used or manipulated in any way, for the production of a substance by the cell, for example the expression by the cell of a gene, a DNA or RNA sequence, a protein or an enzyme. Host cells can further be used for screening or other assays, as described infra.

A “coding sequence” or a sequence “encoding” an expression product, such as a RNA, polypeptide, protein, or enzyme, is a nucleotide sequence that, when expressed, results in the production of that RNA, polypeptide, protein, or enzyme, i.e. the nucleotide sequence encodes an amino acid sequence for that polypeptide, protein or enzyme. A coding sequence for a protein may include a start codon (usually ATG) and a stop codon.

The term “gene”, also called a “structural gene” means a DNA sequence that codes for or corresponds to a particular sequence of amino acids which comprise all or part of one or more proteins or enzymes, and may or may not include regulatory DNA sequences, such as promoter sequences, which determine for example the conditions under which the gene is expressed.

A “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease S1), as well as protein binding domains (consensus sequences) responsible for the binding of RNA polymerase.

In vitro or in vivo expression of F-spondin, a neurexin protein, an APP or an APLP, or any other proteins whose specific interactions are characterized herein, may be controlled by any promoter/enhancer element known in the art, but these regulatory elements must be functional in the host selected for expression. Promoters that may be used to control gene expression include, but are not limited to, cytomegalovirus (CMV) promoter (U.S. Pat. Nos. 5,385,839 and 5,168,062), the SV40 early promoter region (Benoist and Chambon, Nature 1981;290:304-310), the promoter contained in the 3 long terminal repeat of Rous sarcoma virus (Yamamoto et al., Cell 1980;22:787-797), the herpes thymidine kinase promoter (Wagner et al. Proc. Natl. Acad. Sci. U.S.A. 1981;78:1441-1445), the regulatory sequences of the metallothionein gene (Brinster et al., Nature 1982;296:39-42); prokaryotic expression vectors such as the β-lactamase promoter (Villa-Kamaroff et al. Proc. Natl. Acad. Sci. U.S.A. 1978;75:3727-3731), or the tac promoter (DeBoer et al., Proc. Natl. Acad. Sci. U.S.A. 1983;80:21-25); see also “Useful proteins from recombinant bacteria” in Scientific American 1980;242:74-94; promoter elements from yeast or other fungi such as the Gal 4 promoter, the ADC (alcohol dehydrogenase) promoter, PGK (phosphoglycerol kinase) promoter, alkaline phosphatase promoter; and the animal transcriptional control regions, which exhibit tissue specificity and have been utilized in transgenic animals: elastase I gene control region which is active in pancreatic acinar cells (Swift et al. Cell 1984;38:639-646; Ornitz et al. Cold Spring Harbor Symp. Quant. Biol. 1986;50:399-409; MacDonald, Hepatology 1987;7:425-515); insulin gene control region which is active in pancreatic beta cells (Hanahan, Nature 1985;315:115-122), albumin gene control region which is active in liver (Pinkert et al., Genes and Devel. 1987;1:268-276), alpha-fetoprotein gene control region which is active in liver (Krumlauf et al., Mol. Cell. Biol. 1985;5:1639-1648; Hammer et al., Science 1987;235:53-58), alpha 1-antitrypsin gene control region which is active in the liver (Kelsey et al., Genes and Devel. 1987;1:161-171).

A coding sequence is “under the control” or “operatively associated with” of transcriptional and translational control sequences in a cell when RNA polymerase transcribes the coding sequence into mRNA, which is then trans-RNA spliced (if it contains introns) and translated into the protein encoded by the coding sequence.

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g. the resulting protein, may also be said to be “expressed” by the cell. An expression product can be characterized as intracellular, extracellular or secreted. The term “intracellular” means something that is inside a cell. The term “extracellular” means something that is outside a cell. A substance is “secreted” by a cell if it appears in significant measure outside the cell, from somewhere on or inside the cell.

The term “transfection” means the introduction of a foreign nucleic acid into a cell. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. The introduced gene or sequence may also be called a “cloned” or “foreign” gene or sequence, may include regulatory or control sequences, such as start, stop, promoter, signal, secretion, or other sequences used by a cell's genetic machinery. The gene or sequence may include nonfunctional sequences or sequences with no known function. A host cell that receives and expresses introduced DNA or RNA has been “transformed” and is a “transformant” or a “clone.” The DNA or RNA introduced to a host cell can come from any source, including cells of the same genus or species as the host cell, or cells of a different genus or species.

The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence. Vectors include plasmids, phages, viruses, etc.; they are discussed in greater detail below.

Vectors typically comprise the DNA of a transmissible agent, into which foreign DNA is inserted. A common way to insert one segment of DNA into another segment of DNA involves the use of enzymes called restriction enzymes that cleave DNA at specific sites (specific groups of nucleotides) called restriction sites. A “cassette” refers to a DNA coding sequence or segment of DNA that codes for an expression product that can be inserted into a vector at defined restriction sites. The cassette restriction sites are designed to ensure insertion of the cassette in the proper reading frame. Generally, foreign DNA is inserted at one or more restriction sites of the vector DNA, and then is carried by the vector into a host cell along with the transmissible vector DNA. A segment or sequence of DNA having inserted or added DNA, such as an expression vector, can also be called a “DNA construct.” A common type of vector is a “plasmid”, which generally is a self-contained molecule of double-stranded DNA, usually of bacterial origin, that can readily accept additional (foreign) DNA and which can readily introduced into a suitable host cell. A plasmid vector often contains coding DNA and promoter DNA and has one or more restriction sites suitable for inserting foreign DNA. Coding DNA is a DNA sequence that encodes a particular amino acid sequence for a particular protein or enzyme. Promoter DNA is a DNA sequence that initiates, regulates, or otherwise mediates or controls the expression of the coding DNA. Promoter DNA and coding DNA may be from the same gene or from different genes, and may be from the same or different organisms. A large number of vectors, including plasmid and fungal vectors, have been described for replication and/or expression in a variety of eukaryotic and prokaryotic hosts. Non-limiting examples include pKK plasmids (Clonetech), pUC plasmids, pET plasmids (Novagen, Inc., Madison, Wis.), pRSET or pREP plasmids (Invitrogen, San Diego, Calif.), or pMAL plasmids (New England Biolabs, Beverly, Mass.), and many appropriate host cells, using methods disclosed or cited herein or otherwise known to those skilled in the relevant art. Recombinant cloning vectors will often include one or more replication systems for cloning or expression, one or more markers for selection in the host, e.g. antibiotic resistance, and one or more expression cassettes.

The term “expression system” means a host cell and compatible vector under suitable conditions, e.g. for the expression of a protein coded for by foreign DNA carried by the vector and introduced to the host cell. Expression systems may include mammalian host cells and vectors. Suitable cells include PC12 cells, COS cells, CHO cells, Hela cells, 293 and 293T (human kidney cells), mouse primary myoblasts, and NIH 3T3 cells. Alternatively, an insect expression system, e.g. using a baculovirus vector, can be employed. The present invention also contemplates yeast and bacterial expression systems.

The term “heterologous” refers to a combination of elements not naturally occurring. For example, heterologous DNA refers to DNA not naturally located in the cell, or in a chromosomal site of the cell. Preferably, the heterologous DNA includes a gene foreign to the cell. A heterologous expression regulatory element is an element operatively associated with a different gene than the one it is operatively associated with in nature. In the context of the present invention, a gene is heterologous to the vector DNA in which it is inserted for cloning or expression, and it is heterologous to a host cell containing such a vector, in which it is expressed, e.g. a CHO cell.

The terms “mutant” and “mutation” mean any detectable change in genetic material, e.g. DNA, or any process, mechanism, or result of such a change. This includes gene mutations, in which the structure (e.g. DNA sequence) of a gene is altered, any gene or DNA arising from any mutation process, and any expression product (e.g. protein or enzyme) expressed by a modified gene or DNA sequence. The term “variant” may also be used to indicate a modified or altered gene, DNA sequence, enzyme, cell, etc., i.e. any kind of mutant.

“Sequence-conservative variants” of a polynucleotide sequence are those in which a change of one or more nucleotides in a given codon position results in no alteration in the amino acid encoded at that position. Sequence conservative variants encoding any of the proteins described herein may be useful in various expression systems, e.g. to incorporate preferred codons in the coding sequence so as to increase expression efficiency, or to incorporate a restriction site to facilitate manipulation of the coding sequence without altering the amino acid sequence.

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids with similar properties are well known in the art. For example, arginine, histidine and lysine are hydrophilic-basic amino acids and may be interchangeable. Similarly, isoleucine, a hydrophobic amino acid, may be replaced with leucine, methionine or valine. Such changes are expected to have little or no effect on the apparent molecular weight or isoelectric point of the protein or polypeptide. Amino acids other than those indicated as conserved may differ in a protein or enzyme so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide or enzyme which has at least 60% amino acid identity as determined by BLAST or FASTA algorithms, preferably at least 75%, most preferably at least 85%, and even more preferably at least 90%, and which has the same or substantially similar properties or functions as the native or parent protein or enzyme to which it is compared. Finally, for purposes of the invention, a functional-conservative variant includes a truncated or other form of the protein that retains its function, such as a truncated F-spondin, neurexin, an APP or APLP peptide.

Similarly, in a particular embodiment, two amino acid sequences are “substantially homologous” or “substantially similar” when greater than 80% of the amino acids are identical, or greater than about 90% are similar (functionally identical). Preferably, the similar or homologous sequences are identified by alignment using, for example, the GCG (Genetics Computer Group, Program Manual for the GCG Package, Version 7, Madison, Wis.) pileup program, or any of the programs described above (BLAST, FASTA), and Clustal W analysis (MacVector). Sequence comparison algorithms can also be found at a bioinformatics website (bioinformatics.html)@nwfsc.noaa.gov on the Worldwide Web (www).

A nucleic acid molecule is “hybridizable” to another nucleic acid molecule, such as a cDNA, genomic DNA, or RNA, when a single stranded form of the nucleic acid molecule can anneal to the other nucleic acid molecule under the appropriate conditions of temperature and solution ionic strength. See Sambrook et al., supra. The conditions of temperature and ionic strength determine the “stringency” of the hybridization. High stringency hybridization conditions correspond to the highest Tₘ, e.g. 50% formamide, 5× or 6×SCC. SCC is a 0.15M NaCl, 0.015M Na-citrate. Hybridization requires that the two nucleic acids contain complementary sequences, although depending on the stringency of the hybridization, mismatches between bases are possible. The appropriate stringency for hybridizing nucleic acids depends on the length of the nucleic acids and the degree of complementation, variables well known in the art. The greater the degree of similarity or homology between two nucleotide sequences, the greater the value of Tₘ for hybrids of nucleic acids having those sequences. The relative stability (corresponding to higher Tₘ) of nucleic acid hybridizations decreases in the following order: RNA:RNA, DNA:RNA, DNA:DNA. For hybrids of greater than 100 nucleotides in length, equations for calculating Tₘ have been derived. See Sambrook et al., supra, 9.50-9.51). For hybridization with shorter nucleic acids, i.e. oligonucleotides, the position of mismatches becomes more important, and the length of the oligonucleotide determines its specificity. See Sambrook et al. supra, 11.7-11.8). A minimum length for a hybridizable nucleic acid is at least about 10 nucleotides; preferably at least about 15 nucleotides; and more preferably the length is at least about 20 nucleotides.

In a specific embodiment, the term “standard hybridization conditions” refers to a Tₘ of 55° C., and utilizes conditions as set forth above. In a preferred embodiment, the Tₘ is 60° C.; in a more preferred embodiment, the Tₘ is 65° C. In a specific embodiment, “high stringency” refers to hybridization and/or washing conditions at 68° C. in 0.2×SSC, at 42° C. in 50% formamide, 4×SSC, or under conditions that afford levels of hybridization equivalent to those observed under either of these two conditions.

As used herein, the term “oligonucleotide” refers to a nucleic acid, generally of at least 10, preferably at least 15, and more preferably at least 20 nucleotides, preferably no more than 100 nucleotides, that is hybridizable to a genomic DNA molecule, a cDNA molecule, or an mRNA molecule encoding a gene, mRNA, cDNA, or other nucleic acid of interest. Oligonucleotides can be labeled, e.g. with ³²P-nucleotides or nucleotides to which a label, such as biotin, has been covalently conjugated. In one embodiment, a labeled oligonucleotide can be used as a probe to detect the presence of a nucleic acid. In another embodiment, oligonucleotides (one or both of which may be labeled) can be used as PCR primers, either for cloning full length or a fragment of a F-spondin or neurexin or an APP or APLP protein or polypeptide. Generally, oligonucleotides are prepared synthetically, preferably on a nucleic acid synthesizer. Accordingly, oligonucleotides can be prepared with non-naturally occurring phosphoester analog bonds, such as thioester bonds, etc.

The present invention provides antisense nucleic acids (including ribozymes), which may be used to inhibit expression of one or more specific proteins. An “antisense nucleic acid” is a single stranded nucleic acid molecule which, on hybridizing under cytoplasmic conditions with complementary bases in an RNA or DNA molecule, inhibits the latter's role. If the RNA is a messenger RNA transcript, the antisense nucleic acid is a countertranscript or mRNA-interfering complementary nucleic acid. As presently used, “antisense” broadly includes RNA-RNA interactions, RNA-DNA interactions, ribozymes and RNase-H mediated arrest. Antisense nucleic acid molecules can be encoded by a recombinant gene for expression in a cell (e.g. U.S. Pat. Nos. 5,814,500; 5,811,234), or alternatively they can be prepared synthetically (e.g. U.S. Pat. No. 5,780,607).

Specific non-limiting examples of synthetic oligonucleotides envisioned for this invention include oligonucleotides that contain phosphorothioates, phosphotriesters, methyl phosphonates, short chain alkyl, or cycloalkl intersugar linkages or short chain heteroatomic or heterocyclic intersugar linkages. Most preferred are those with CH₂—NH—O—CH₂, CH₂—N(CH₃)—O—CH₂, CH₂—O—N(CH₃)—CH₂, CH₂—N(CH₃)—N(CH₃)—CH₂ and O—N(CH₃)—CH₂—CH₂ backbones (where phosphodiester is O—PO₂—O—CH₂). U.S. Pat. No. 5,677,437 describes heteroaromatic olignucleoside linkages. Nitrogen linkers or groups containing nitrogen can also be used to prepare oligonucleotide mimics (U.S. Pat. Nos. 5,792,844 and 5,783,682). U.S. Pat. No. 5,637,684 describes phosphoramidate and phosphorothioamidate oligomeric compounds. Also envisioned are oligonucleotides having morpholino backbone structures (U.S. Pat. No. 5,034,506). In other embodiments, such as the peptide-nucleic acid (PNA) backbone, the phosphodiester backbone of the oligonucleotide may be replaced with a polyamide backbone, the bases being bound directly or indirectly to the aza nitrogen atoms of the polyamide backbone (Nielsen et al. Science 1991;254:1497). Other synthetic oligonucleotides may contain substituted sugar moieties comprising one of the following at the 2′ position: OH, SH, SCH₃, F, OCN, O(CH₂)ₙNH₂ or O(CH₂)ₙCH₃ where n is from 1 to about 10; C₁ to C₁₀ lower alkyl, substituted lower alkyl, alkaryl or aralkyl; Cl; Br, CN; CF₃; OCF₃; O-; S-, or N-alkyl; O-, S-, or N-alkenyl; SOCH₃; SO₂CH₃; ONO₂; NO₂; N₃; NH₂; heterocycloalkyl; heterocycloalkaryl; aminoalkylamino; polyalkylamino; substituted silyl; a fluorescein moiety; an RNA cleaving group; a reporter group; an intercalator; a group for improving the pharmacokinetic properties of an oligonucleotide; or a group for improving the pharmacodynamic properties of an oligonucleotide, and other substituents having similar properties. Oligonucleotides may also have sugar mimetics such as cyclobutyls or other carbocyclics in place of the pentofuranosyl group. Nucleotide units having nucleosides other than adenosine, cytidine, guanosine, thymidine and uridine, such as inosine, may be used in an oligonucleotide molecule.

### Vectors

A wide variety of host/expression vector combinations may be employed in expressing DNA sequences encoding F-spondin, a neurexin peptide, an APP or APLP and any intracellular signal transduction factors. Useful expression vectors, for example, may consist of segments of chromosomal, non-chromosomal and synthetic DNA sequences. Suitable vectors include derivatives of SV40 and known bacterial plasmids, e.g. E. coli plasmids col E1, pCR1, pBR322, pMal-C2, pET, pGEX (Smith et al., Gene 1988;67:31-40), pMB9 and their derivatives, plasmids such as RP4; phage DNAS, e.g. the numerous derivatives of phage 1, e.g. NM989, and other phage DNA, e.g. M13 and filamentous single stranded phage DNA; yeast plasmids such as the 2 m plasmid or derivatives thereof; vectors useful in eukaryotic cells, such as vectors useful in insect or mammalian cells; vectors derived from combinations of plasmids and phage DNAs, such as plasmids that have been modified to employ phage DNA or other expression control sequences; and the like.

A vector can be introduced in vivo in a non-viral vector, e.g. by lipofection, with other transfection facilitating agents (peptides, polymers, etc.), or as naked DNA. Synthetic cationic lipids can be used to prepare liposomes for in vivo transfection, with targeting in some instances (Felgner et. al., Proc. Natl. Acad. Sci. U.S.A. 1987;84:7413-7417; Felgner and Ringold, Science 1989;337:387-388; see Mackey et al., Proc. Natl. Acad. Sci. U.S.A. 1988;85:8027-8031; Ulmer et al., Science 1993;259:1745-1748). Useful lipid compounds and compositions for transfer of nucleic acids are described in International Patent Publications WO95/18863 and WO96/17823, and in U.S. Pat. No. 5,459,127. Other molecules are also useful for facilitating transfection of a nucleic acid in vivo, such as a cationic oligopeptide (e.g. International Patent Publication WO95/21931), peptides derived from DNA binding proteins (e.g. International Patent Publication WO96/25508), or a cationic polymer (e.g. International Patent Publication WO95/21931). Recently, a relatively low voltage, high efficiency in vivo DNA transfer technique, termed electrotransfer, has been described (Mir et al., C. P. Acad. Sci. 1998;321:893; WO 99/01157; WO 99/01158; WO 99/01175). DNA vectors for gene therapy can be introduced into the desired host cells by methods known in the art, e.g. electroporation, microinjection, cell fusion, DEAE dextran, calcium phosphate precipitation, use of a gene gun (bioballistic transfection), or use of a DNA vector transporter (see e.g. Wu et al., J. Biol. Chem. 1992;267:963-967; Wu and Wu, J. Biol. Chem. 1988;263:14621-14624; Hartmut et al., Canadian Patent Application No. 2,012,311, filed Mar. 15, 1990; Williams et al., Proc. Natl. Acad. Sci. USA 1991;88:2726-2730). Receptor-mediated DNA delivery approaches can also be used (Curiel et al., Hum. Gene Ther. 1992;3:147-154; Wu and Wu, J. Biol. Chem. 1987;262:4429-4432). U.S. Pat. Nos. 5,580,859 and 5,589,466 disclose delivery of exogenous DNA sequences, free of transfection facilitating agents, in a mammal.

Also useful are viral vectors, such as lentiviruses, retroviruses, herpes viruses, adenoviruses, adeno-associated viruses, vaccinia virus, baculovirus, and other recombinant viruses with desirable cellular tropism. Thus, a gene encoding a functional protein or polypeptide (as set forth above) can be introduced in vivo, ex vivo, or in vitro using a viral vector or through direct introduction of DNA. Expression in targeted tissues can be effected by targeting the transgenic vector to specific cells, such as with a viral vector or a receptor ligand, or by using a tissue-specific promoter, or both. Targeted gene delivery is described in International Patent Publication WO 95/28494, published October 1995.

Viral vectors commonly used for in vivo or ex vivo targeting and therapy procedures are DNA-based vectors and retroviral vectors. Methods for constructing and using viral vectors are known in the art (see e.g. Miller and Rosman, BioTechniques, 1992;7:980-990). Preferably, the viral vectors are replication defective, i.e. they are unable to replicate autonomously in the target cell. In general, the genome of the replication defective viral vectors which are used within the scope of the present invention lack at least one region which is necessary for the replication of the virus in the infected cell. These regions can either be eliminated (in whole or in part), be rendered non-functional by any technique known to a person skilled in the art. These techniques include the total removal, substitution (by other sequences, in particular by the inserted nucleic acid), partial deletion or addition of one or more bases to an essential (for replication) region. Such techniques may be performed in vitro (on the isolated DNA) or in situ, using the techniques of genetic manipulation or by treatment with mutagenic agents. Preferably, the replication defective virus retains the sequences of its genome that are necessary for encapsidating the viral particles.

DNA viral vectors include an attenuated or defective DNA virus, such as but not limited to herpes simplex virus (HSV), papillomavirus, Epstein Barr virus (EBV), adenovirus, adeno-associated virus (AAV), and the like. Defective viruses, which entirely or almost entirely lack viral genes, are preferred. Defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector (Kaplitt et al., Molec. Cell. Neurosci. 1991;2:320-330), defective herpes virus vector lacking a glyco-protein L gene (Patent Publication RD 371005 A), or other defective herpes virus vectors (International Patent Publication No. WO 94/21807, published Sep. 29, 1994; International Patent Publication No. WO 92/05263, published Apr. 2, 1994); an attenuated adenovirus vector, such as the vector described by Stratford-Perricaudet et al. (J. Clin. Invest. 1992;90:626-630; see also Le Gal La Salle et al., Science 1993;259:988-990); and a defective adeno-associated virus vector (Samulski et al., J. Virol. 1987;61:3096-3101; Samulski et al., J. Virol. 1989;63:3822-3828; Lebkowski et al., Mol. Cell. Biol. 1988;8:3988-3996).

Various companies produce viral vectors commercially, including but by no means limited to Avigen, Inc. (Alameda, Calif.; AAV vectors), Cell Genesys (Foster City, Calif.; retroviral, adenoviral, AAV vectors, and lentiviral vectors), Clontech (retroviral and baculoviral vectors), Genovo, Inc. (Sharon Hill, Pa.; adenoviral and AAV vectors), Genvec (adenoviral vectors), IntroGene (Leiden. Netherlands; adenoviral vectors), Molecular Medicine (retroviral, adenoviral, AAV, and herpes viral vectors), Norgen (adenoviral vectors), Oxford BioMedica (Oxford, United Kingdom; lentiviral vectors), and Transgene (Strasbourg, France; adenoviral, vaccinia, retroviral, and lentiviral vectors).

Preferably, for in vivo administration, e.g. to create a transient transgenic animal, an appropriate immunosuppressive treatment is employed in conjunction with the viral vector, e.g. adenovirus vector, to avoid immuno-deactivation of the viral vector and transfected cells. For example, immunosuppressive cytokines, such as interleukin-10 (IL-10), interleukin-12 (IL-12), interferon-γ (IFN-γ), or anti-CD4 antibody, can be administered to block humoral or cellular immune responses to the viral vectors (see, e.g. Wilson and Kay, Nature Medicine 1995;1:887-889). In that regard, it is advantageous to employ a viral vector that is engineered to express a minimal number of antigens.

### Synthetic Peptides

The term “peptide” is used in its broadest sense to refer to a compound of two or more subunit amino acids, amino acid analogs or peptidomimetics. The subunits may be linked by peptide bonds. In another embodiment, the subunit may be linked by other the bonds, e.g. ester, ether, etc. As used herein the term “amino acid” refers to either natural and/or unnatural or synthetic amino acids, including glycine and both the D or L optical isomers, and amino acid analogs and peptidomimetics. Thus, peptides of the invention may comprise D-amino acids, a combination of D- and L-amino acids, and various “designer” amino acids (e.g. β-methyl amino acids, Cα-methyl amino acids, and Nα-methyl amino acids, etc.) to convey special properties to peptides in the library. Additionally, by assigning specific amino acids at specific coupling steps, peptide libraries with α-helices, β turns, β sheets, γ-turns, and cyclic peptides can be generated. A peptide of three or more amino acids is commonly called an oligopeptide if the peptide chain is short. If the peptide chain is long, the peptide is commonly called a polypeptide or a protein.

The coupling of the amino acids may be accomplished by techniques familiar to those in the art and provided, for example, in Stewart and Young, 1984, Solid Phase Synthesis, Second Edition, Pierce Chemical Co., Rockford, Ill. Amino acids used for peptide synthesis may be standard Boc (Nα-amino protected Nα-t-butyloxycarbonyl) amino acid resin with the standard deprotecting, neutralization, coupling and wash protocols of the original solid phase procedure of Merrifield (J. Am. Chem. Soc. 1963;85:2149-2154), or the base-labile Nα-amino protected 9-fluorenylmethoxycarbonyl (Fmoc) amino acids first described by Carpino and Han (J. Org. Chem. 1972;37:3403-3409). Both Fmoc and Boc α-amino protected amino acids can be obtained from Fluka, Bachem, Advanced Chemtech, Sigma, Cambridge Research Biochemical, Bachem, or Peninsula Labs or other chemical companies familiar to those who practice this art. In addition, the method of the invention can be used with other Nα-protecting groups that are familiar to those skilled in this art. Many methods of activation may be used in the practice of the invention and include, for example, preformed symmetrical anhydrides (PSA), preformed mixed anhydride (PMA), acid chlorides, active esters, and in situ activation of the carboxylic acid, as set forth in Fields and Noble, “Solid phase peptide synthesis utilizing 9-fluorenylmethoxycarbonyl amino acids”, Int. J. Pept. Protein Res. 1990;35:161-214. Solid phase peptide synthesis may be accomplished by techniques familiar to those in the art and provided, for example, in Stewart and Young, 1984. Solid Phase Synthesis, Second Edition, Pierce Chemical Co., Rockford, Ill.; Fields and Noble, Int. J. Pept. Protein Res. 1990;35:161-214, or using automated synthesizers, such as sold by ABS.

The completeness of coupling should be assessed. Those skilled in the art would be familiar with the well known quantitative monitoring tests such as ninhydrin (the Kaiser test), picric acid, 2,4,6-trinitro-benzenesulfonic (TNBS), fluorescamine, and chloranil, which are based on reagent reaction with free amino groups to produce a chromophoric compound. If imino acids (e.g. Pro and Hyp) are used, isatin monitoring is a preferred method. Fields and Noble, supra. Quantification of reaction completeness may be monitored during the course of the reaction, e.g. as described by Salisbury et al. (International Patent Publication No. WO91/03485).

If the coupling reaction is incomplete as determined by this test, the reaction can be forced to completion by several methods familiar to those in the art, including (a) a second coupling using a one to five fold excess of protected amino acid, (b) an additional coupling using different or additional solvents (e.g. trifluoroethane), or (c) the addition of chaotropic salts, e.g. NaCIO₄ or LiBr (Klis and Stewart, 1990, “Peptides: Chemistry, Structure and Biology,” Rivier and Marshall, eds., ESCOM Publ., p. 904-906).

The following non-classical amino acids may be incorporated in peptides of the invention to introduce particular conformational motifs: 1,2,3,4-tetrahydroisoquinoline-3-carboxylate (Kazmierski et al., J. Am. Chem. Soc. 1991; 113:2275-2283); (2S,3S)-methyl-phenylalanine, (2S,3R)-methyl-phenylalanine, (2R,3S)-methyl-phenylalanine and (2R,3R)-methyl-phenylalanine (Kazmierski and Hruby, 1991, Tetrahedron Lett.); 2-aminotetrahydronaphthalene-2-carboxylic acid (Landis, 1989, Ph.D. Thesis, University of Arizona); hydroxy-1,2,3,4-tetrahydroisoquinoline-3-carboxylate (Miyake et al., J. Takeda Res. Labs. 1989;43:53-76); b-carboline (D and L) (Kazmierski, 1988, Ph.D. Thesis, University of Arizona); HIC (histidine isoquinoline carboxylic acid) (Zechel et al., Int. J. Pep. Protein Res. 1991;43); and HIC (histidine cyclic urea) (Dharanipragada).

The following amino acid analogs and peptidomimetics may be incorporated to induce or favor specific secondary structures: LL-Acp (LL-3-amino-2-propenidone-6-carboxylic acid), a β-turn inducing dipeptide analog (Kemp et al., J. Org. Chem. 1985;50:5834-5838); β-sheet inducing analogs (Kemp et al., Tetrahedron Lett. 1988;29:5081-5082); β-turn inducing analogs (Kemp et al., Tetrahedron Lett. 1988;29:5057-5060); μ-helix inducing analogs (Kemp et al., Tetrahedron Lett. 1988;29:4935-4938); γ-turn inducing analogs (Kemp et al., J. Org. Chem. 1989;54:109:115); and analogs provided by the following references: Nagai and Sato, Tetrahedron Lett. 1985;26:647-650; DiMaio et al., 1989, J. Chem. Soc. Perkin Trans. p. 1687; also a Gly-Ala turn analog (Kahn et al., Tetrahedron Lett. 1989;30:2317); amide bond isostere (Jones et al., Tetrahedron Lett. 1988;29:3853-3856); tretrazol (Zabrocki et al., J. Am. Chem. Soc. 1988;110:5875-5880); DTC (Samanen et al., Int. J. Protein Pep. Res. 1990;35:501:509); and analogs taught in Olson et al., J. Am. Chem. Sci. 1990;112:323-333 and Garvey et al., J. Org. Chem. 1990;56:436.

### F-Spondin Polypeptides

As noted above, the term F-spondin polypeptides includes full-length F-spondin, F-spondin fusion proteins, and F-spondin fragments that can bind to APP or its homologs and modulate APP-mediated signaling.

Full-length F-spondin. cDNAs encoding F-spondin have been isolated from numerous species including rat (GenBank Acc. No. NM 172067) and human (GenBank Acc. No. NM 006108). The predicted protein encoded by the human gene is more than 90% identical to the rat gene product indicating an extremely high degree of sequence conservation. This implies that the human and rat gene products are functionally very similar.

The F-spondin open reading frame predicts a novel protein of 807 amino acids. At the N terminus, F-spondin contains a cleavable signal peptide followed by a 200 bp region that is homologous to reelin, a protein that binds to the LDL superfamily of receptors and also to amyloid plaques. See U.S. Pat. No. 6,323,177 and Wirths et al., Neurosci. Lett. 2001;316:145-148. The central region of F-spondin contains a prototypical “spondin” domain, while the C-terminal region contains six thrombospondin type 1 repeats. See Feinstein et al., Development 1999;126:3637-3648.

F-spondin fusion proteins. Various chimeric constructs prepared by fusing an F-spondin amino acid sequence with a non-F-spondin amino acid sequence (or “heterologous” sequence) are contemplated as well. Preferably, the heterologous sequence provides some functional activity. In a specific embodiment, the heterologous sequence acts as an immunoaffinity tag that does not impair the ability of F-spondin to specifically bind to an APP or an APLP.

For example, F-spondin can be tagged with an N-terminal or C-terminal tag, such as Myc, FLAG, glutathione-S-transferase (GST), an immunoglobulin or an immunoglobulin fragment such as the Fc domain, or another such tag for detectable antibody binding or immunoprecipitation. F-spondin can also be fused with a reporter protein, such as alkaline phosphatase, horseradish peroxidase, β-lactamase, β-galactosidase, luciferase, green fluorescent protein, and the like. In specific embodiments, exemplified infra, F-spondin is fused to immunoglobulins, myc or GST. In preferred embodiments, the F-spondin Ig-fusion proteins comprise the portions of the F-spondin molecule set forth in SEQ ID NOS:2, 4, 6, 8, 10 or 12, which are encoded by the nucleotides of SEQ ID NO:1, 3, 5, 7, 9, and 11, respectively.

Alternatively, a signal sequence can be substituted for the endogenous signal sequence for more efficient processing into the rough endoplasmic reticulum, Golgi, and cell membrane. Similarly, an expression tag, such as an α-mating factor sequence for yeast expression, or residual amino acid residues from a recombinant construct, may be present.

In yet another embodiment, a chromatographic tag or handle can be joined to F-spondin. For example, a polyhistidine sequence permits purification on a nickel chelation column.

F-spondin protein fragments and deletion mutants. Preliminary analysis has indicated that truncated F-spondin peptides are capable of binding to an APP. Various F-spondin peptides and deletion constructs can be prepared for testing in the screen of the invention. Such testing can be used, for example, to delimit the smallest region of F-spondin capable of binding to an APP or an APLP. This can be carried out by a combination of deletion mutagenesis analysis and peptide synthesis (as described above).

These peptides may act as agonists for the receptor or they may block binding of full-length F-spondin to an APP, thereby preventing cleavage of the APP and activation of APP-dependent transactivation pathways. This allows identification of peptides with unique properties.

Truncated F-spondin proteins may be N-terminal, C-terminal, or they may contain internal fragments comprising some of the F-spondin repeats. In preferred embodiments, the truncated F-spondin proteins are polypeptides of between 50 and 800 amino acids, encoded by nucleic acids of between 150 and 2400 base pairs, that comprise all or part of the spondin domain of F-spondin. Examples of truncated F-spondin proteins are the Ig-F-spondins 2-7 of FIG. 2A. The complete nucleotide and amino acid of the F-spondin portions of Ig-F-spondins 1-6 are set forth in FIG. 2E and in SEQ ID NOS:1-12. In particularly preferred embodiments of the instant invention, the F-spondin fragments are the polypeptides set forth in SEQ ID NOS:2, 4, 6, 8, and 12. Because some of the truncated forms of F-spondin depicted in FIG. 2A can still bind to APP₆₉₅ (e.g. Ig-F-spondins 2-4 and 6), the TSR regions do not appear to be essential for APP binding.

Similar approaches may be employed for the creation and testing of neurexin peptides.

### APP and APLP Peptides

F-spondin has been shown to bind to APP₆₉₅. See Examples, infra. F-spondin has been shown to bind also to APLP1 and APLP 2. See Examples, infra. Thus, the deletion mutagenesis techniques described above, which have been used to discern the regions of F-spondin that specifically interact with APP, also may be used to delimit the region of F-spondin that specifically interacts with APLP. Similarly, these same techniques may be used to determine the regions of APP and APLP that specifically interact with F-spondin. These methods may allow identification of peptides with unique properties. For example, truncated APP or APLP peptides or fragments may specifically block the action of F-spondin. Such APP or APLP peptides or fragments will likely be found in the central extracellular region (CER). See Examples, infra. These molecules may be useful as competitive inhibitors or antagonists of F-spondin binding to an APP or an APLP, and thus be useful as potential therapeutic agents for AD.

Similar approaches may be employed for the identification of competitive inhibitors of the binding of neurexin proteins to an APP or an APLP.

### Screening Assays

The present invention further provides various screening assays for modulators of F-spondin/APP, F-spondin/APLP, neurexin/APP and neurexin/APLP interactions. The assays of the invention are particularly advantageous by permitting rapid evaluation of cellular response. Biological assays, which often depend on cell growth, survival, or some other response, require substantial amounts of time and resources to evaluate. By directly detecting the specific binding interactions between the two binding partners or the specific signal modulated by the binding of the two binding partners, the present invention obviates the need for more tedious, time consuming and expensive biological assays. Furthermore, such assays can often be performed with very small amounts of material.

In general, a screening assay of the invention makes use of the cells expressing an APP or an APLP, either alone or in combination with other cellular proteins and reporter gene constructs as described above, F-spondin or neurexin polypeptides as described above, and a candidate compound for testing.

The present invention contemplates screens for small molecule compounds, including ligand analogs and mimics, as well as screens for natural compounds that bind to and agonize, antagonize, stabilize or otherwise modulate APP- or APLP-mediated signal transduction in vivo. Such agonists or antagonists may, for example, interfere with the binding of agents to the extracellular domains of an APP or an APLP, with the cleavage of an APP or an APLP, or with the interaction of AICD and cellular proteins such as Fe65, Tip60 and Mints/X11. For example, natural products libraries can be screened using assays of the invention for such molecules. Antagonists of F-spondin binding may act by binding to a primary F-spondin binding site on APP or APLP, thereby inhibiting the subsequent binding of F-spondin, or may act by binding to secondary sites on APP or APLP, whereafter the conformation of the primary F-spondin binding site on APP or APLP is altered in a way that prevents binding of F-spondin.

As used herein, the term “compound” refers to any molecule or complex of more than one molecule that affects APP-mediated signal transduction. The present invention contemplates screens for synthetic small molecule compounds, chemical compounds, chemical complexes, and salts thereof as well as screens for natural products, such as plant extracts or materials obtained from fermentation broths. Other molecules that can be identified using the screens of the invention include proteins and peptide fragments, peptides, nucleic acids and oligonucleotides (particularly triple-helix-forming oligonucleotides), carbohydrates, phospholipids and other lipid derivatives, steroids and steroid derivatives, prostaglandins and related arachadonic acid derivatives, etc. Of particular interest are peptides and peptide mimetics that correspond to the domains of F-spondin, the neurexins, an APP or an APLP that mediate the binding of each molecule to the other molecule in the binding pair. Particularly preferred are peptides or peptide mimetics that correspond to the spondin domain of F-spondin.

One approach to identifying such compounds uses recombinant bacteriophage to produce large libraries. Using the “phage method” (Scott and Smith, Science 249:386-390, 1990; Cwirla, et al., Proc. Natl. Acad. Sci., 87:6378-6382, 1990; Devlin et al., Science, 49:404-406, 1990), very large libraries can be constructed (10⁶-10⁸ chemical entities). A second approach uses primarily chemical methods, of which the Geysen method (Geysen et al., Molecular Immunology 23:709-715, 1986; Geysen et al. J. Immunologic Method 102:259-274, 1987; and the method of Fodor et al. (Science 251:767-773, 1991) are examples. Furka et al. (14th International Congress of Biochemistry, Volume #5, Abstract FR:013, 1988; Furka, Int. J. Peptide Protein Res. 37:487-493, 1991), Houghton (U.S. Pat. No. 4,631,211, issued December 1986) and Rutter et al. (U.S. Pat. No. 5,010,175, issued Apr. 23, 1991) describe methods to produce a mixture of peptides that can be tested as agonists or antagonists.

In another aspect, synthetic combinatorial libraries (Needels et al., Proc. Natl. Acad. Sci. USA 90:10700-4, 1993; Ohlmeyer et al., Proc. Natl. Acad. Sci. USA 90:10922-10926, 1993; Lam et al., International Patent Publication No. WO 92/00252; Kocis et al., International Patent Publication No. WO 9428028) and the like can be used to screen for compounds according to the present invention.

Test compounds are screened from large libraries of synthetic or natural compounds. Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (NC), or are readily producible. Additionally, natural and synthetically produced libraries and compounds are readily modified through conventional chemical, physical, and biochemical means (Blondelle et al., Tib Tech, 14:60, 1996).

### Binding Assays

A cell-based assay can be used to screen a few or large numbers of peptides or chemical compounds for their ability to modulate the binding of F-spondin or a neurexin to an APP or an APLP molecule. Mammalian or insect cells expressing an APP or APLP are produced in large scale using the expression constructs described above. Suitable mammalian cells include, but are not limited to, 293T, Jurkat, Hela, COS, CHO, MEF, or NIH3T3 cells. Insect cells may be SF9 or derivatives of this line. Cells are seeded on microplate dishes, rinsed with phosphate buffered saline (PBS) and overlaid with medium containing F-spondin or a neurexin polypeptide as defined above. Cells are then washed with PBS and solubilized in the well by the addition of a detergent-containing buffer such as TXB buffer supplemented with protease inhibitors. This method can be modified to use fixed, rather than live cells in the binding assay. Microplate dishes are centrifuged to remove the insoluble material, and the soluble cellular proteins are analyzed to detect the presence of F-spondin or a neurexin.

The method of detection will vary depending upon the particular form of APP ligand used in the binding assay. For example, if an F-spondin-alkaline phosphatase fusion protein is employed, detection can be accomplished using a colorimetric system to measure the enzymatic activity of alkaline phosphatase. Alternatively, immunodetection can be performed using antibodies against an epitope tag or against F-spondin or neurexin. Secondary antibodies conjugated to a fluorophor such as FITC or Texas Red, or antibodies conjugated to an enzyme such as alkaline phosphatase or horseradish peroxidase can be employed. The staining is then analyzed using a fluorimeter or a spectrophotometer. Additionally, F-spondin or neurexin can be radiolabeled, for example by iodination with ¹²⁵I or ³²P to allow detection by autoradiography or scintillation detection, or F-spondin or neurexin could be biotinylated to allow detection by streptavidin-linked reagents.

In addition to cell-based assay systems, cell-free binding assays can be used to screen for agonists, antagonists or other modulators of the interactions among F-spondin or the neurexins and an APP or an APLP. Purified proteins or cell extracts can be used in which one of the partners is immobilized on beads or in microtiter wells and the other is used in soluble form. The same approaches to detection of the interaction using fusion proteins, enzyme-linked assays, antibodies and radioisotopes as described above. An example of this approach is provided below in the Examples. Alternatively, a BioCore binding assay system can be employed to identify binding interactions in a cell-free system. This will allow the rapid analysis of compounds or natural products in a high throughput screen that does not require cell culture.

The binding assays of the invention can be adapted for high-throughput screens, e.g. using automated systems. Preferably such systems are microprocessor controlled. These systems automatically add and remove reagents from a large number of individual reactions, usually in a microwell array, and are often adapted to read results as well (e.g. by detecting fluorescence or some other output signal). Both cell and cell-free binding assays can be adapted to the high-throughput format.

The F-spondin, neurexin, APP or APLP peptides described above can be produced for testing in binding assays to determine interesting properties of such peptides. These properties may include, but are not limited to: 1) F-spondin or neurexin peptides that bind to and prevent cleavage of APP or APLP; 2) F-spondin or neurexin peptides that bind to APP or APLP but do not prevent cleavage; 3) F-spondin or neurexin peptides that prevent the binding of APP or APLP by other ligands; and 4) non-cleavable APP or APLP peptides that block the binding of F-spondin or neurexin peptides to endogenous APP or APLP.

The binding assay may also be used to investigate the effects of other agents on the interaction between F-spondin and neurexin and an APP or an APLP molecule. The regions of the APP or APLP polypeptide responsible for this effect may be discerned by deletion analysis as described above. After defining the minimum peptide region necessary for the interactions among F-spondin or a neurexin protein and an APP or APLP, mutagenesis studies can identify peptides that exhibit higher binding affinities.

The analysis described above will allow the identification of amino acid sequences that are critical for the binding of F-spondin and a neurexin protein to an APP or an APLP. Based on this information, it is possible to determine the structural features of these binding regions, separately and as complexes, using techniques such as X-ray crystallography, neutron diffraction, nuclear magnetic resonance spectrometry together with bioinformatic approaches. The knowledge of the structure of each of the binding interfaces involved in the interaction between F-spondin and a neurexin protein with an APP or APLP molecule will facilitate the rational design or identification of agonists and antagonists of F-spondin, neurexin, APP and APLP molecules. In particular, synthetic peptides or peptidomimetics, as described above, can be prepared based on this information.

### Methods for Detecting Signals

The present invention provides numerous methods for detecting signals, including but not limited to detecting signals from transactivated genes, especially reporter genes. Alternatively, the protein products of endogenous genes that are transactivation targets of the AICD complex, such as CD82, can be detected directly or the CD82 gene can be modified so that it has reporter activity, e.g. through the expression of a CD82/green fluorescent protein (GFP) reporter gene. Reporter genes for use in the invention encode detectable proteins, including, but are by no means limited to, chloramphenicol transferase (CAT), β-galactosidase (β-gal), luciferase, green fluorescent protein (GFP), alkaline phosphatase, and other genes that can be detected, e.g. immunologically (by antibody assay).

The best-characterized intracellular signaling molecule created by the cleavage of APP is the APP intracellular domain (AICD). Cao and Südhof, Science 2001;293:115-120. Because binding of F-spondin to APP blocks APP cleavage and hence the formation of AICD and Aβ, the assay of the instant invention can be used to identify molecules that interfere with APP-mediated signal transduction. These molecules may act as agonists or antagonists of the interactions between F-spondin and an APP or an APLP. Such modulators may be useful in preventing formation of Aβ and hence in the prevention of Alzheimer's disease.

### Modulation of APP Activity

The present invention provides for modulating the activity of an APP or APLP molecule. For example, the binding of APP or APLP by a variety of extracellular ligands including F-spondin or neurexins may be beneficial to neurons or other cell types. This binding may result in reduction of plaque formation, synaptic degeneration or neurodegeneration in AD or other neurodegenerative diseases. Because F-spondin is critical for neuronal migration, other possible utilities of the present invention include the identification of therapeutic agents based on the F-spondin/APP or F-spondin/APLP binding interactions described herein for use in the treatment of neurodevelopmental disorders or in regeneration of peripheral nerves. See Burstyn-Cohen et al., J. Neurosci. 1998;18:8875-8885. Similarly, because F-spondin and related molecules play a role in angiogenesis (Terai et al., J. Cell Physiol. 2001;188:394-402), agents that modulate F-spondin binding may be useful in intervening in this biological process. Such agents therefore may be useful therapeutically in the treatment of cancer or other pathological states in which angiogenesis has been implicated.

### In Vivo Testing Using Transgenic Animals

Transgenic mammals can be prepared for evaluating the molecular mechanisms of F-spondin, and particularly human F-spondin/APP- or APLP-induced signaling. Such mammals provide excellent models for screening or testing drug candidates. Thus, mammals transgenic for human F-spondin, an APP, an APLP, or various combinations thereof, may be prepared using “knock-in” technologies. Such mammals may be useful in evaluating the molecular biology of this system in greater detail than is possible with human subjects. It is also possible to evaluate compounds or diseases on “knockout” animals, e.g. to identify a compound that can compensate for a defect in F-spondin or APP activity. Both technologies permit manipulation of single units of genetic information in their natural position in a cell genome and to examine the results of that manipulation in the background of a terminally differentiated organism.

A “knock-in” mammal is a mammal in which an endogenous gene is substituted with a heterologous gene (Roemer et al., New Biol. 1991;3:331). Preferably, the heterologous gene is “knocked-in” to a locus of interest, thereby linking the heterologous gene expression to transcription from the appropriate promoter. This can be achieved by homologous recombination, by transposons (Westphal and Leder, Curr Biol 1997;7:530), using mutant recombination sites (Araki et al., Nucleic Acids Res 1997;25:868) or by PCR (Zhang and Henderson, Biotechniques 1998;25:784).

A “knock-out mammal” is a mammal (e.g. mouse) that contains within its genome a specific gene that has been inactivated by the method of gene targeting. See e.g. U.S. Pat. Nos. 5,777,195 and 5,616,491. A knock-out mammal includes both a heterozygote knock-out (i.e. one defective allele and one wild-type allele) and a homozygous mutant.

Preparation of knock-in and knock-out mammals requires first introducing a nucleic acid construct that will be used to suppress expression of a particular gene into an undifferentiated cell type termed an embryonic stem cell. This cell is then injected into a mammalian embryo. A mammalian embryo with an integrated cell is then implanted into a foster mother for the duration of gestation. Zhou et al. (Genes and Development, 1995;9:2623-34) describes PPCA knock-out mice.

The term “knock-out” refers to partial or complete suppression of the expression of at least a portion of a protein encoded by an endogenous DNA sequence in a cell. The term “knock-out construct” refers to a nucleic acid sequence that is designed to decrease or suppress expression of a protein encoded by endogenous DNA sequences in a cell. The nucleic acid sequence used as the knock-out construct is typically comprised of (1) DNA from some portion of the gene (exon sequence, intron sequence, and/or promoter sequence) to be suppressed and (2) a marker sequence used to detect the presence of the knock-out construct in the cell. The knock-out construct is inserted into a cell, and integrates with the genomic DNA of the cell in such a position so as to prevent or interrupt transcription of the native DNA sequence. Such insertion usually occurs by homologous recombination (i.e. regions of the knock-out construct that are homologous to endogenous DNA sequences hybridize to each other when the knock-out construct is inserted into the cell and recombine so that the knock-out construct is incorporated into the corresponding position of the endogenous DNA). The knock-out construct nucleic acid sequence may comprise 1) a full or partial sequence of one or more exons and/or introns of the gene to be suppressed, 2) a full or partial promoter sequence of the gene to be suppressed, or 3) combinations thereof. Typically, the knock-out construct is inserted into an embryonic stem cell (ES cell) and is integrated into the ES cell genomic DNA, usually by the process of homologous recombination. This ES cell is then injected into, and integrates with, the developing embryo.

The phrases “disruption of the gene” and “gene disruption” refer to insertion of a nucleic acid sequence into one region of the native DNA sequence (usually one or more exons) and/or the promoter region of a gene so as to decrease or prevent expression of that gene in the cell as compared to the wild-type or naturally occurring sequence of the gene. By way of example, a nucleic acid construct can be prepared containing a DNA sequence encoding an antibiotic resistance gene which is inserted into the DNA sequence that is complementary to the DNA sequence (promoter and/or coding region) to be disrupted. When this nucleic acid construct is then transfected into a cell, the construct will integrate into the genomic DNA. Thus, many progeny of the cell will no longer express the gene at least in some cells, or will express it at a decreased level, as the DNA is now disrupted by the antibiotic resistance gene.

Generally, for homologous recombination, the DNA will be at least about 1 kilobase (kb) in length and preferably 3-4 kb in length, thereby providing sufficient complementary sequence for recombination when the knock-out construct is introduced into the genomic DNA of the ES cell (discussed below).

Included within the scope of this invention is a mammal in which two or more genes have been knocked out or knocked in, or both. Such mammals can be generated by repeating the procedures set forth herein for generating each knock-out construct, or by breeding to mammals, each with a single gene knocked out, to each other, and screening for those with the double knock-out genotype.

Regulated knock-out animals can be prepared using various systems, such as the tet-repressor system (see U.S. Pat. No. 5,654,168) or the Cre-Lox system (see U.S. Pat. Nos. 4,959,317 and 5,801,030).

In another series of embodiments, transgenic animals are created in which (i) a human F-spondin gene or an APP gene, or both, is stably inserted into the genome of the transgenic animal; and/or (ii) the endogenous F-spondin or an APP, or both, genes are inactivated and replaced with their human counterparts. See e.g. Coffman, Semin. Nephrol. 1997;17:404; Esther et al., Lab. Invest. 1996;74:953; Murakami et al., Blood Press. Suppl. 1996;2:36. Such animals can be treated with candidate compounds and monitored for cognitive impairment, neurodegeneration, or efficacy of a candidate therapeutic compound.

### Gene Therapy to Modulate APP Activity

A gene encoding a truncated or mutant F-spondin, neurexin, an APP or APLP protein or polypeptide characterized using a screen of the invention can be introduced in vivo, ex vivo, or in vitro using a viral or a non-viral vector, e.g. as discussed above. Expression in targeted tissues can be effected by targeting the transgenic vector to specific cells, such as with a viral vector or a receptor ligand, or by using a tissue-specific promoter, or both. Targeted gene delivery is described in International Patent Publication WO 95/28494, published October 1995.

Preferably, for in vivo administration, an appropriate immunosuppressive treatment is employed in conjunction with the viral vector, e.g. adenovirus vector, to avoid immune-mediated destruction of the transfected cells or inactivation of the viral vector. For example, immunosuppressive cytokines, such as interleukin-12 (IL-12), interferon-γ (IFN-γ), or anti-CD4 antibody, can be administered to block humoral or cellular immune responses to the viral vectors (see, e.g. Wilson and Kay, Nature Medicine 1995;1:887-889). In that regard, it is advantageous to employ a viral vector that is engineered to express a minimal number of antigens.

Adenovirus vectors. Adenoviruses are eukaryotic DNA viruses that can be modified to efficiently deliver a nucleic acid of the invention to a variety of cell types in vivo, and has been used extensively in gene therapy protocols. Various serotypes of adenovirus exist. Of these serotypes, preference is given to using type 2 or type 5 human adenoviruses (Ad 2 or Ad 5) or adenoviruses of animal origin (see WO94/26914). Those adenoviruses of animal origin that can be used within the scope of the present invention include adenoviruses of canine, bovine, murine (example: Mav1, Beard et al., Virology 1990;75:81), ovine, porcine, avian, and simian (example: SAV) origin. Preferably, the adenovirus of animal origin is a canine adenovirus, more preferably a CAV2 adenovirus (e.g. Manhattan or A26/61 strain (ATCC VR-800). Various replication defective adenovirus and minimum adenovirus vectors have been described for gene therapy (WO94/26914, WO95/02697, WO94/28938, WO94/28152, WO94/12649, WO95/02697 WO96/22378). The replication defective recombinant adenoviruses according to the invention can be prepared by any technique known to the person skilled in the art (Levrero et al., Gene 1991;101:195; EP 185 573; Graham, EMBO J. 1984;3:2917; Graham et al., J. Gen. Virol. 1977;36:59). Recombinant adenoviruses are recovered and purified using standard molecular biological techniques, which are well known to one of ordinary skill in the art.

Adeno-associated viruses. The adeno-associated viruses (AAV) are DNA viruses of relatively small size that can integrate, in a stable and site-specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. The use of vectors derived from the AAVs for transferring genes in vitro and in vivo has been described. See e.g. WO 91/18088; WO 93/09239; U.S. Pat. Nos. 4,797,368 and 5,139,941; EP 488 528). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line which is infected with a human helper virus (for example an adenovirus). The AAV recombinants that are produced are then purified by standard techniques.

Retrovirus vectors. In another embodiment the gene can be introduced in a retroviral vector, e.g. as described in Anderson et al., U.S. Pat. No. 5,399,346; Mann et al., Cell 1983;33:153; Temin et al., U.S. Pat. No. 4,650,764; Temin et al., U.S. Pat. No. 4,980,289; Markowitz et al., J. Virol. 1988;62:1120; Temin et al., U.S. Pat. No. 5,124,263; EP 453242, EP178220; Bernstein et al. Genet. Eng. 1985;7:235; McCormick, BioTechnology 1985;3:689; International Patent Publication No. WO 95/07358; and Kuo et al., Blood 1993;82:845. The retroviruses are integrating viruses that infect dividing cells. The retrovirus genome includes two LTRs, an encapsidation sequence and three coding regions (gag, pol and env). In recombinant retroviral vectors, the gag, pol and env genes are generally deleted, in whole or in part, and replaced with a heterologous nucleic acid sequence of interest. These vectors can be constructed from different types of retrovirus, such as MoMuLV (“murine Moloney leukemia virus”), MSV (“murine Moloney sarcoma virus”), HaSV (“Harvey sarcoma virus”), SNV (“spleen necrosis virus”), RSV (“Rous sarcoma virus”), and Friend virus. Suitable packaging cell lines have been described in the prior art, in particular the cell line PA317 (U.S. Pat. No. 4,861,719); the ΨCRIP cell line (WO 90/02806) and the GP+envAM-12 cell line (WO 89/07150). In addition, the recombinant retroviral vectors can contain modifications within the LTRs for suppressing transcriptional activity as well as extensive encapsidation sequences which may include a part of the gag gene (Bender et al., J. Virol. 1987;61:1639). Recombinant retroviral vectors are purified by standard techniques known to those having ordinary skill in the art.

Retrovirus vectors can also be introduced by recombinant DNA viruses, which permit one cycle of retroviral replication and amplifies transfection efficiency. See WO 95/22617, WO 95/26411, WO 96/39036, and WO 97/19182.

Lentivirus vectors. In another embodiment, lentiviral vectors are can be used as agents for the direct delivery and sustained expression of a transgene in several tissue types, including brain, retina, muscle, liver and blood. The vectors can efficiently transduce dividing and nondividing cells in these tissues, and maintain long-term expression of the gene of interest. See Naldini, Curr. Opin. Biotechnol., 1998;9:457-63; Zufferey, et al., J. Virol. 1998;72:9873-80). Lentiviral packaging cell lines are available and known generally in the art. They facilitate the production of high-titer lentivirus vectors for gene therapy. An example is a tetracycline-inducible VSV-G pseudotyped lentivirus packaging cell line which can generate virus particles at titers greater than 106 IU/ml for at least 3 to 4 days (Kafri et al., J. Virol. 1999;73:576-584). The vector produced by the inducible cell line can be concentrated as needed for efficiently transducing nondividing cells in vitro and in vivo.

Non-viral vectors. In another embodiment, the vector can be introduced in vivo using any of the non-viral vector strategies discussed above in connection with “Vectors”, e.g. by lipofection, with other transfection facilitating agents (peptides, polymers, etc.), electroporation, electrotransfer, microinjection, cell fusion, DEAE dextran, calcium phosphate precipitation, use of a gene gun, or use of a DNA vector transporter.

The following nonlimiting examples serve to further illustrate the present invention.

### EXAMPLES

### Example 1

Materials and Methods

Plasmids. Vectors encoding various parts of human APP₆₉₅ or F-spondin (ATCC 2190694) were generated by subcloning the corresponding PCR fragments into pCMV-Ig9 (Ushkaryov et al., 1994, J. Biol. Chem. 269:11987-11992), pGEX-KG, or pCMV5 (plasmid names with residue numbers from APP₆₉₅). pCMVIg-APP.1=residues 1-678; pCMVIg-APP.2=1-205; pGEX-CAPPD=286-557, pCMV-APP as previously described by Cao and Südhof, 2001, Science 293:115-120), pCMV-APPΔ1=deletion of residues 36-289 with insertion of Pro-Trp residues; pCMV-APPΔ2=deletion of residues 288-493 with insertion of Thr-Arg residues; pCMVIg-F spondin.1=residues 1-807 (full-length); pCMVIg-F spondin.2=1-501; pCMVIg-F spondin.3=1-614, pCMVIg-F spondin.4=1-754; pCMVIg-F spondin.5=1-225; pCMVIg-F spondin.6=1-442; pCMVIg-F spondin.7=443-807. Full-length myc-tagged F-spondin was generated by subcloning NotI-ClaI PCR fragments into pcDNA4-His/myc B. Vectors encoding human full-length Mindin (ATCC 5183118; Feinstein et al., 1999, Development 126:3637-3648) were generated by subcloning EcoRI-SalI fragment to pCMVIg9 vector and EcoRI-XhoI fragment to pcDNA4-His/myc A vector.

Generation of brain membrane extracts. Twenty frozen rat brains (Pelfreeze) were homogenized in 200 ml 0.32 M sucrose, 5 mM HEPES-NaOH pH 7.4, and 0.1 mM EDTA containing a standard protease inhibitor mix (0.1 g/L PMSF, 104 mg/L leupeptin, and aprotinin, and 1 mg/L pepstatin A). The homogenate was centrifuged at low speed (800×g for 15 min) to remove debris, and the supernatant was centrifuged (100,000×g for 1 h) to yield a crude membrane pellet that was homogenized in buffer A (20 mM HEPES-NaOH pH 7.4, 150 mM NaCl, 2 mM CaCl2, 2 mM MgCl2 with the standard protease inhibitor mix). Subsequently, an equal volume of buffer B (buffer A containing 2% Triton X-100) was added for extraction (3 hr at 4° C.), and insoluble material was removed by centrifugation (100,000× g for 1 h).

Affinity chromatography on immobilized GST- or Ig-fusion proteins. These studies were performed essentially as described (Ichtchenko et al., 1995, Cell 81:435-443; Hata et al., Nature 366:347-351). Brain membrane extract was precleared by incubation (2 h at 4° C.) with glutathione agarose and incubated overnight at 4° C. with immobilized GST-CAPPD on glutathione agarose beads preequilibrated with buffer B. Beads were washed with buffer B and serially eluted with 2 ml of buffer B containing 0.3 M NaCl, 0.5 M NaCl, 1.0 M NaCl, or 1.0 M NaCl, 10 mM EGTA, and 5 mM EDTA (instead of 2 mM CaCl2). Eluted proteins were analyzed by SDS-PAGE and Coomassie blue staining. Bound proteins were identified by liquid chromatography/mass spectroscopy of tryptic fragments. For pulldown assays, the medium from COS cells transfected with pcDNA4-His/myc-F spondin or pcDNA-His/myc-Mindin (collected 48-72 h post transfection) was adjusted to (final concentrations) 10 mM HEPES-NaOH pH 7.4, 1 mM EGTA, 1% Triton X-100, proteinase inhibitors were added, and the supernatant was precleared with immobilized GST orIg-C. The treated medium was then incubated overnight at 4° C. with GST or GST-CAPPD immobilized on glutathione agarose or with various Ig-APP fusion proteins immobilized on protein-A Sepharose. Glutathione agarose or Protein A beads were washed 4-5× with buffer B, and examined by SDS-PAGE and immunoblotting. COS cells that were transfected with pCMV-APP, pCMV-APPΔ1, pCMV-APPΔ2, or pCMV-APLPs were harvested in PBS 48 h post-transfection, membrane proteins were solubilized in buffer B, and the cell lysate was incubated overnight at 4° C. with Protein A Sepharose containing Ig-F spondins, Ig-Mindin, or control Ig-C fusion protein. Protein A beads were washed with buffer B 4-5×, and resuspended in SDS-PAGE sample buffer.

APP cleavage in transfected cells by BACE 1. HEK293 cells were co-transfected in 12 well plates using FuGENE reagent with APP alone, APP with BACE1, or combinations of APP and BACE1 with Ig-F spondin or Ig-C. APP fragments were examined by immunoblotting and quantitated using ¹²⁵I-labeled secondary antibodies (Amersham) with PhosphorImager (Molecular Dynamics) detection (Rosahl et al., Nature 375:488-493).

Transactivation assays. HEK293 cells were co-transfected in 12 well plates using Lipofectamine 2000 with pCMV-APP, pCMV-Tip60, pCMV-Fe65 and reporter plasmids pG5E1B-luc and pCMV-LacZ alone, or with Ig-F spondin, or Ig-neurexin 1β, or Ig-Mindin, or Ig-SynCAM, or Ig-neurexin1β-3 or Ig-neurexin1α-1 or control Ig-C. Transactivation assays were performed as described (Cao and Südhof, 2001, Science 293:115-120; Biederer et al., 2002, J. Neurosci. 22:7340-7351). The luciferase activity was standardized by the β-galactosidase activity as a control for transfection efficiency.

### Example 2

Identification of F-spondin as a potential APP ligand. N-terminally, APP is composed of a signal peptide (SP), a cysteine-rich domain (CRD), a zinc-binding motif, acidic sequence regions and a Kunitz domain (FIG. 1A). The center of APP is occupied by a large domain that contains no cysteine residues (referred to as central APP domain=CAPPD) and a short linker sequence that includes the cleavage sites for α- and β-secretases (FIG. 1A). C-terminally, APP contains a transmembrane region and a cytoplasmic tail (FIG. 1A). Nonneuronal APP contains an alternatively spliced Kunitz domain.

To search for APP ligands, a recombinant GST-fusion protein was produced that contained the central extracellular conserved domain of APP (CAPPD; see FIG. 1A). Since the CAPPD has no cysteines, it lacks disulfide bonds and can be produced in bacteria. The CAPPD-GST fusion protein was immobilized on glutathione-Sepharose for affinity chromatography experiments with membrane proteins that were solubilized from rat brain with 1% Triton X-100. Immobilized GST was used as a negative control. Bound proteins were eluted with high salt, and identified by mass spectroscopy F-spondin as a major component of the proteins bound to CAPPD.

To confirm the potential interaction of F-spondin with APP, COS cells were transfected with a gene encoding a fusion protein comprising the Fc-region of human immunoglobulin and various portions of the extracellular sequence of APP. The constructs examined, shown in FIG. 1A, include Ig-fusion proteins of the entire extracellular region or CRD alone (Ig-APP.1 or Ig-APP.2 respectively), a GST-CAPPD fusion protein, and expression vectors that encode full-length APP or APP in which the CRD or part of the CAPPD were deleted marked by dashed lines (PCMV-APPΔ1 or pCMV-APPΔ2, respectively).

The purified Ig-APP fusion protein, immobilized on protein A-Sepharose, was examined to determine whether it could pull down myc-tagged full-length F-spondin that was also produced in transfected COS cells (FIG. 1B). An Ig-fusion protein that contains only a few N-terminal residues of neurexin 1α in addition to the immunoglobulin moiety (Ig-C; FIG. 1A) was used as an negative control. Binding of recombinant secreted F-spondin to the immobilized Ig-APP fusion protein was examined in the presence and absence of Ca²⁺, because many extracellular binding domains are stabilized by structural Ca²⁺-binding. In these studies, it was observed that Ig-APP but not Ig-C captured F-spondin, and that F-spondin was bound only in the presence of Ca²⁺ (FIG. 1B).

An Ig-fusion protein that includes only the N-terminal domains of APP (Ig-APP.2) also was examined in these studies, but no binding between this protein and F-spondin was observed (FIG. 1B). However, the isolated central conserved APP domain (CAPPD), immobilized as a bacterially expressed GST-fusion protein, did bind to F-spondin in a Ca²⁺-dependent manner similar to the Ig-fusion protein containing the full-length extracellular sequences of APP (FIG. 1B).

### Example 3

Validation of F-spondin Binding to APP. A series of F-spondin Ig-fusion proteins that include different parts of F-spondin (FIG. 2A) were constructed and employed to perform pulldowns of recombinant full-length APP695 expressed in COS cells (FIG. 2B) or of endogenous brain APP. In these studies, APP695 was solubilized with 1% Triton X-100 from transfected COS cells, and bound to immobilized Ig-F spondin proteins containing full-length or parts of F-spondin. These studies demonstrated that immobilized F-spondin specifically retained recombinant and native brain APP, and that the N-terminal reelin domain and the central spondin-specific region of F-spondin were essential for binding APP, whereas the C-terminal thrombospondin repeats were not required (FIG. 2B).

### Example 4

Specificity of APP binding by F-spondin. Expression of deletion constructs of APP revealed that deletion of the N-terminal cysteine-rich growth-factor like domain (CRD; APPΔ1) did not abolish binding, whereas a partial deletion of the central APP domain (CAPPD; APPΔ2) blocked binding (FIG. 2C). APP is closely related to the APP-like proteins 1 and 2 (APLP1 and APLP2; Wasco et al., 1992, Proc. Natl. Acad. Sci. USA 89:10758-10762; Sprecher et al., 1993, Biochemistry 32:4481-4486; Wasco et al., 1993, Nature Genetics 5:95-99; Slunt et al., 1994, J. Biol. Chem. 269:2637-2644), and the CAPPD is particularly well conserved among these proteins. If binding of F-spondin to APP is specific, APLPs also should bind. Indeed, all three proteins were similarly captured by immobilized F-spondin (FIG. 2D). Viewed together, these findings indicate that the central APP domain (CAPPD) of APP directly binds to F-spondin.

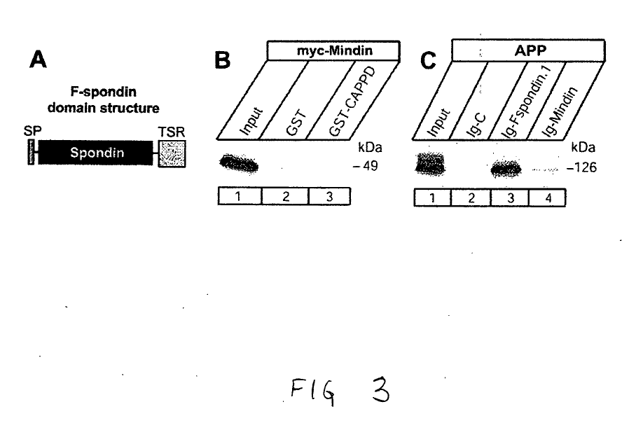

A protein related to F-spondin called Mindin has recently been characterized. Miyamoto et al., 2001, Arch. Biochem. Biophys. 390:93-100. Mindin contains a spondin-like sequence and a single thrombospondin repeat, but lacks a reelin domain (FIG. 3A). To test whether Mindin might bind to APP, experiments similar to those described in FIGS. 1 and 2 were performed with myc-tagged Mindin (FIG. 3B) or with a Ig-Mindin fusion protein (FIG. 3C). In contrast to F-spondin, no Mindin binding was observed in either assay configuration, suggesting that Mindin does not bind to APP.

### Example 5

F-spondin inhibits APP cleavage by BACE 1, the primary β-secretase. A key feature of APP is that it is digested by α- and β-secretases which cleave APP at a site C-terminal of the CAPPD (FIG. 1A). To test whether binding of F-spondin alters APP cleavage, a gene encoding BACE 1, the enzyme that mediates β-secretase activity (Sinha et al., 1999, Nature 40-42), was co-transfected with a gene encoding APP. When only APP was expressed in HEK293 cells, APP C-terminal fragments (CTFs) were barely detectable at a low steady-state level when APP cleavage was analyzed by immunoblotting with an antibody specific for the C-terminal of APP (FIG. 4A, lanes 1-3; experiments are carried out in triplicates for quantifications). However, when a gene encoding BACE 1 was cotransfected with a gene encoding APP, the steady-state level of CTFs dramatically increased (FIG. 4A, lanes 4-6). After BACE 1 cleavage, two closely migrating CTFs were observed that may correspond to the two major BACE 1 cleavage sites in APP (Sinha et al., 1999, Nature 402:537-540; Vassar et al., 1999, Science 286:735-741). When a gene encoding control Ig-fusion protein (Ig-C) was cotransfected together with genes encoding APP and BACE 1, a small decrease was observed in both APP and the CTFs (FIG. 4A, lanes 7-9), probably because co-transfection of a third plasmid dilutes the cellular transcription/translation machinery. However, when a gene encoding a full-length Ig-F spondin fusion protein was co-transfected with genes encoding APP and BACE 1, a dramatic decline in level of CTFs was observed, indicating that F-spondin inhibits BACE 1-dependent APP cleavage (FIG. 4A, lanes 10-12). The decrease in CTFs by F-spondin was confirmed in quantitations of full-length APP and the CTFs in the transfected cells using ¹²⁵I-labeled secondary antibodies (FIG. 4B). In these studies, relative levels of full-length APP and of both CTFs were quantified using ¹²⁵I-labeled secondary antibodies and phosphoimager detection. Data shown in FIG. 4B are means±SEMs derived by dividing for each sample the signal for CTFβ1 or CTFβ2 by the APP signal. These studies demonstrated that F-spondin decreased the CTFs of APP by ˜70-80% (corrected for the amount of full-length APP present to control for co-transfection effects).

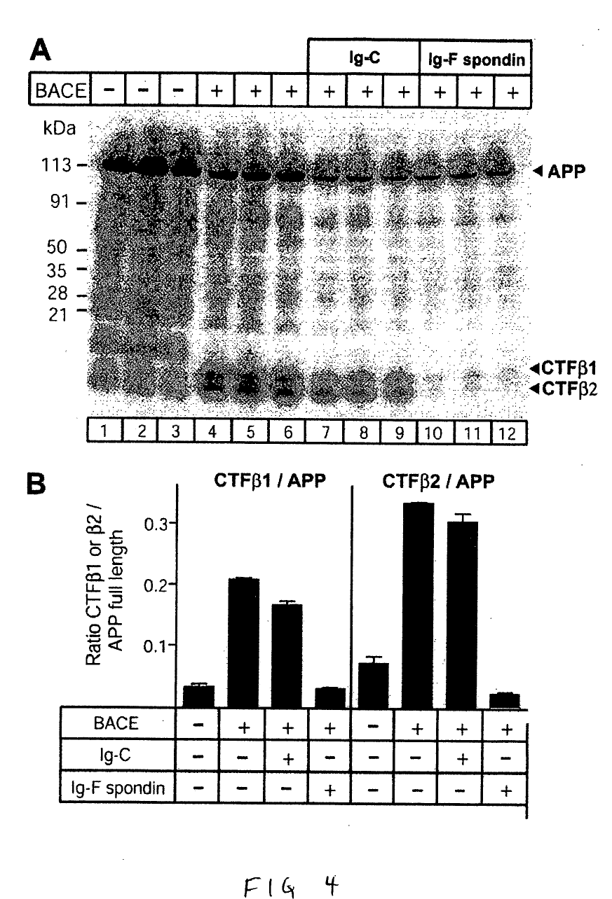

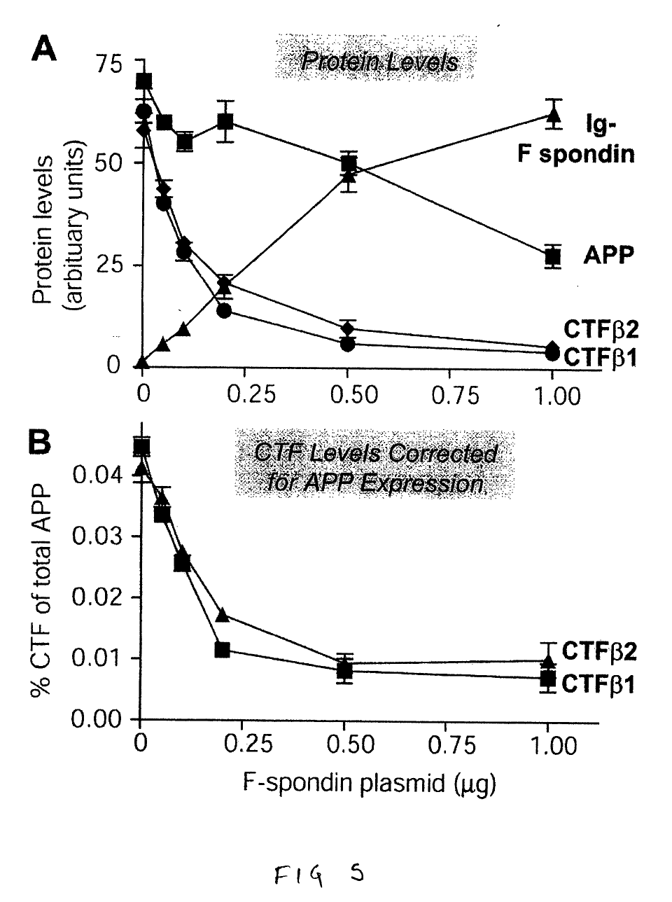

To determine whether the effect of F-spondin on APP cleavage was dose-dependent, similar transfection experiments were performed with increasing amounts of a plasmid encoding F-spondin, and the levels of F-spondin protein, APP proteins and CTFs were quantified. In these studies, increasing amounts of Ig-F spondin plasmid were cotransfected with constant amounts of APP and BACE 1. The levels of full-length APP and the CTFs of APP and of F-spondin were quantified by immunoblotting and are shown in FIG. 5A as arbitrary units. Data shown are means±SEMs from a representative experiment (n=3) independently repeated multiple times.

As expected, transfection of increasing amounts of F-spondin plasmid led to a dose-dependent increase in F-spondin protein (FIG. 5A). In addition, a moderate decrease in full-length APP was observed, presumably because of competition between transfected plasmids for transcription. Transfection of <0.25 μg F-spondin plasmid inhibited CTF production ˜75%, but had <20% effect on APP levels (FIG. 5A). Correcting the CTF levels for those of full-length APP confirmed that the drop in CTFs was not a simple reflection of the small decrease in APP, but was due to a large decline in APP cleavage by relatively low levels of F-spondin (FIG. 5B).

### Example 6

F-spondin impairs APP-dependent transactivation of Gal4-Tip60 transcription. Previous studies suggested that the AICD of APP functions in transcriptional activation by binding to the adaptor protein Fe65 that in turns binds to the chromosome remodeling factor Tip60 (Cao and Südhof, 2001, Science 293:115-120). Unmodified APP strongly transactivates Gal4-Tip60 mediated transcription by a mechanism that depends on Fe65, probably because the AICD of APP (which binds to Fe65) is released by α-/β- and γ-cleavage of APP and cooperates with Fe65 in transcription.

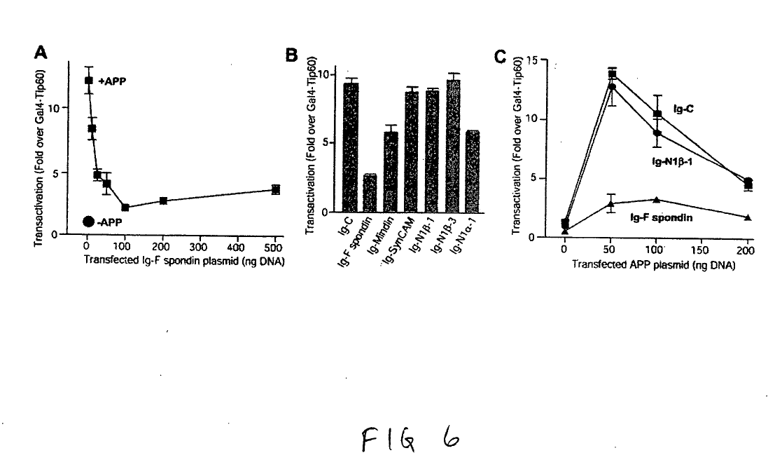

To test whether F-spondin alters the transcriptional activation mediated by APP as an additional, indirect assay for APP cleavage, increasing amounts of a plasmid encoding Ig-F spondin were transfected with a constant amount of plasmids encoding APP and/or Fe65 into HEK293 cells (FIG. 6A). Expression of APP alone greatly stimulated Gal4-Tip60 dependent transactivation as expected (Cao and Südhof, 2001, Science 293:115-120), However, cotransfection of even low amounts of F-spondin plasmid (<100 ng) inhibited transactivation, consistent with an inhibition of cleavage by F-spondin.

To test the specificity of this inhibition, small amounts of plasmids (50 ng DNA) encoding various unrelated Ig-fusion proteins (Ig-C, Ig-F spondin, Ig-Mindin, Ig-SynCAM, and three different Ig-neurexins) were cotransfected with plasmids encoding Gal4-Tip60 and/or Fe65, and the relative level of transactivation by APP was measured in the presence of these Ig-fusion proteins (FIG. 6B). Expression of Ig-F spondin potently inhibited transactivation, while expression of Ig-SynCAM, Ig-N1β-1, and Ig-N1β-3 produced no inhibition of transactivation; expression of Ig-Mindin and Ig-N1α-1 caused an intermediate degree of inhibition.

The intermediate inhibition of transactivation caused by Ig-Mindin and Ig-N1α-1, although significantly less than the inhibition mediated by F-spondin, raised the possibility that the F-spondin dependent inhibition in this assay is not specific, but an indirect effect. Ig-SynCAM, Ig-N1β-1, Ig-N1β-3 may have been unable to inhibit because they were expressed in the wrong ratio with APP. To address this possibility, increasing amounts of APP were cotransfected with a constant amount of Fe65 and of either an Ig-control protein (Ig-C), Ig-neurexin 1β (Ig-N1β-1), or Ig-F spondin. This experiment was designed to control for potential non-specific effects of the immunoglobulin moiety in the Ig-F spondin fusion protein, or for trafficking effects induced by expressing a neuronal cell-surface protein. Increasing concentrations of APP were tested in order to account for the possibility that a protein did not truly inhibit transactivation, but simply shifted the requirement for APP. Indeed, in the presence of Ig-C, APP potentiated transcription in a bell-shaped dose-response curve (FIG. 6C) as described previously (Cao and Südhof, 2001, Science 293:115-120). This bell-shaped dose-response is probably due to the fact that high concentrations of APP are less efficient in stimulating transcription because the overexpressed APP dilutes out expression of the other components. Ig-F spondin greatly inhibited transactivation at all APP levels, whereas Ig-neurexin 1β (Ig-N1β-1) had no effect (FIG. 6C).

Together these data are consistent with the notion that F-spondin, by binding to the extracellular CAPPD of APP, inhibits APP processing and thereby impairs transcriptional transactivation.

### Example 7

Validation of neurexin binding. Preliminary experiments identified neurexin proteins as a second putative ligand for APP. To confirm the interaction of neurexins with APP, recombinant neurexin-Ig fusion proteins were produced and used to and performed pulldown assays of APP₆₉₅ expressed in transfected COS cells using the immobilized Ig-neurexins as an affinity matrix. The full-length extracellular regions of neurexin 1α and 1β specifically bound APP, with the strongest binding observed for neurexin 1β. In contrast to the binding of β-neurexin to neuroligins, which is specific for the spliced-out β-neurexin variants (Ichtchenko et al., Cell 1995;81:435-443), both splice variants of β-neurexin bound to APP. In contrast to the results observed with F-spondin, co-transfection of neurexin 1β with BACE and APP exerted no change in APP cleavage, suggesting that, unlike binding of F-spondin to APP, binding of neurexin to APP has no effect on APP cleavage. These findings indirectly validate the specificity of the effect of F-spondin binding on APP cleavage.

All references cited herein are incorporated by reference in their entirety.

We claim:

1. A composition comprising an isolated F-spondin polypeptide bound to an amyloid-β precursor protein (APP) polypeptide.

2. The composition of claim 1, wherein the APP polypeptide is selected from the group consisting of APP₆₉₅, APP₇₅₁, and APP₇₇₀.

3. The composition of claim 1, wherein the isolated F-spondin polypeptide is a fusion protein.

4. The composition of claim 3, wherein the fusion protein is selected from the group consisting of an immunoglobulin-F-spondin fusion protein, a Myc-F-spondin fusion protein, and a GLT-F-spondin fusion protein.

5. The composition of claim 4, wherein the immunoglobulin-F-spondin fusion protein is an immunoglobulin-F-spondin.1 peptide, wherein the F-spondin of said fusion protein has the amino acid sequence of SEQ ID NO:2.

6. The composition of claim 4, wherein the immunoglobulin-F-spondin fusion protein is the immunoglobulin-F-spondin.2 peptide, wherein the F-spondin of said fusion protein has the amino acid sequence of SEQ ID NO:4.

7. The composition of claim 4, wherein the immunoglobulin-F-spondin fusion protein is the immunoglobulin-F-spondin.3 peptide, wherein the F-spondin of said fusion protein has the amino acid sequence of SEQ ID NO:6.

8. The composition of claim 4, wherein the immunoglobulin-F-spondin fusion protein is the immunoglobulin-F-spondin.4 peptide, wherein the F-spondin of said fusion protein has the amino acid sequence of SEQ ID NO:8.

9. The composition of claim 4, wherein the immunoglobulin-F-spondin fusion protein is the immunoglobulin-F-spondin.6 peptide, wherein the F-spondin of said fusion protein has the amino acid sequence of SEQ ID NO:12.

10. A composition comprising an isolated F-spondin polypeptide bound to an amyloid-β precursor protein-like protein (APLP) polypeptide.

11. The composition of claim 10, wherein the APLP polypeptide is selected from the group consisting of APLP1 and APLP2.

12. The composition of claim 10, wherein the isolated F-spondin polypeptide is a fusion protein.

13. The composition of claim 12, wherein the fusion protein is selected from the group consisting of an immunoglobulin-F-spondin fusion protein, a Myc-F-spondin fusion protein, and a glutathione S-transferase (GST)-F-spondin fusion protein.

14. The composition of claim 13, wherein the immunoglobulin-F-spondin fusion protein is an immunoglobulin-F-spondin.1 peptide, wherein the F-spondin of said fusion protein has the amino acid sequence of SEQ ID NO:2.

15. The composition of claim 13, wherein the immunoglobulin-F-spondin fusion protein is the immunoglobulin-F-spondin.2 peptide, wherein the F-spondin of said fusion protein has the amino acid sequence of SEQ ID NO:4.

16. The composition of claim 13, wherein the immunoglobulin-F-spondin fusion protein is the immunoglobulin-F-spondin.3 peptide, wherein the F-spondin of said fusion protein has the amino acid sequence of SEQ ID NO:6.

17. The composition of claim 13, wherein the immunoglobulin-F-spondin fusion protein is the immunoglobulin-F-spondin.4 peptide, wherein the F-spondin of said fusion protein has the amino acid sequence of SEQ ID NO:8.

18. The composition of claim 13, wherein the immunoglobulin-F-spondin fusion protein is the immunoglobulin-F-spondin.6 peptide, wherein the F-spondin of said fusion protein has the amino acid sequence of SEQ ID NO:12.

19. The composition of claim 1, further comprising a test compound.

20. The composition of claim 10, further comprising a test compound.

21. An isolated nucleic acid encoding a truncated F-spondin protein wherein said nucleic acid: (i) comprises SEQ ID NO:3; and (ii) encodes a protein that binds to APP.

22. An isolated polypeptide encoded by the nucleic acid fragment of claim 21.

23. A method of identifying a compound that modulates the binding of an F-spondin polypeptide to an APP polypeptide comprising measuring binding of an F-spondin polypeptide to an APP polypeptide in the presence and absence of a test compound, wherein modulation of the binding of F-spondin polypeptide to the APP polypeptide is indicative that the test compound is a modulator of the binding of the F-spondin polypeptide to the APP polypeptide.

24. A method of identifying a compound that modulates the binding of an F-spondin polypeptide to an APLP polypeptide comprising measuring the binding of the F-spondin polypeptide to the APLP polypeptide in the presence and absence of a test compound, wherein modulation of the binding of the F-spondin polypeptide to the APLP polypeptide is indicative that the test compound is a modulator of the binding of the F-spondin polypeptide to the APLP polypeptide.

25. A method of modulating the proteolytic cleavage of APP comprising administering a compound that modulates the binding of an F-spondin polypeptide to an APP polypeptide.


SEQUENCE LISTING (12 sequences)

SEQ ID NO: 1 - DNA, 2436 bp, Homo sapiens
ttgggggccgcgaagatgaggctgtccccggcgcccctgaagctgagccggactccggcactgctggccctggcgctgcccctggccgcggcgctggccttctccgacgagaccctggacaaagtgcccaagtcagagggctactgcagccgtatcctgcgcgcccagggcacgcggcgcgagggctacaccgagttcagcctccgcgtggagggcgaccccgacttctacaagccgggaaccagctaccgcgtaacactttcagctgctcctccctcctacttcagaggattcacattaattgccctcagagagaacagagagggtgataaggaagaagaccatgctgggaccttccagatcatagacgaagaagaaactcagtttatgagcaattgccctgttgcagtcactgaaagcactccacggaggaggacccggatccaggtgttttggatagcaccaccagcgggaacaggctgcgtgattctgaaggccagcatcgtacaaaaacgcattatttattttcaagatgagggctctctgaccaagaaactttgtgaacaagattccacatttgatggggtgactgacaaacccatcttagactgctgtgcctgcggaactgccaagtacagactcacattttatgggaattggtccgagaagacacacccaaaggattaccctcgtcgggccaaccactggtctgcgatcatcggaggatcccactccaagaattatgtactgtgggaatatggaggatatgccagcgaaggcgtcaaacaagttgcagaattgggctcacccgtgaaaatggaggaagaaattcgacaacagagtgatgaggtcctcaccgtcatcaaagccaaagcccagtggccagcctggcagcctctcaacgtgagagcagcaccttcagctgaattttccgtggacagaacgcgccatttaatgtccttcctgaccatgatgggccctagtcccgactggaacgtaggcttatctgcagaagatctgtgcaccaaggaatgtggctgggtccagaaggtggtgcaagacctgattccctgggacgctggcaccgacagcggggtgacctatgagtcacccaacaaacccaccattccccaggagaaaatccggcccctgaccagcctggaccatcctcagagtcctttctatgacccagagggtgggtccatcactcaagtagccagagttgtcatcgagagaatcgcacggaagggtgaacaatgcaatattgtacctgacaatgtcgatgatattgtagctgacctggctccagaagagaaagatgaagatgacacccctgaaacctgcatctactccaactggtccccatggtccgcctgcagctcctccacctgtgacaaaggcaagaggatgcgacagcgcatgctgaaagcacagctggacctcagcgtcccctgccctgacacccaggacttccagccctgcatgggccctggctgcagtgacgaagacggccccacctgcaccatgtccgagtggatcacctggtcgccctgcagcatctcctgcggcatgggcatgaggtcccgggagaggtatgtgaagcagttcccggaggacggctccgtgtgcacgctgcccactgaggaaacggagaagtgcacggtcaacgaggagtgctctcccagcagctgcctgatgaccgagtggggcgagtgggacgagtacagcgccacctgcggcatgggcatgaagaagcggcaccgcatgatcaagatgaaccccgcagatggctccatgtgcaaagccgagacatcacaggcagagaagcgcatgatgccagagtgccacaccatcccatgcttgctgtccccatggtccgagtggagtgactgcagcgtgacctgcgggaagggcatgcgaacccgacagcggatgctcaagtctctggcagaacttggagactgcaatgaggatctggagcaggtggagaagtgcatgctccctgaatgccccattgactgtgagctcaccgagtggtcccagtggtcggaatgtaacaagtcatgtgggaaaggccacgtgattcgaacccggatgatccaaatggagcctcagtttggaggtgcaccctgcccagagactgtgcagcgaaaaaagtgccgcatccgaaaatgccttcgaaatccatccatccaaaagctacgctggagggaggcccgagagagccggcggagtgagcagctgaaggaagagtctgaaggggagcagttcccaggttgtaggatgcgcccatggacggcctggtcagaatgcaccaaactgtgcggaggtggaattcaggaacgttacatgactgtaaagaagagattcaaaagctcccagtttaccagctgcaaagacaagaaggagatcagagcatgcaatgttcatccttgt

SEQ ID NO: 2 - protein, 807 aa, Homo sapiens
MRLSPAPLKLSRTPALLALALPLAAALAFSDETLDKVPKSEGYCSRILRAQGTRREGYTEFSLRVEGDPDFYKPGTSYRVTLSAAPPSYFRGFTLIALRENREGDKEEDHAGTFQIIDEEETQFMSNCPVAVTESTPRRRTRIQVFWIAPPAGTGCVILKASIVQKRIIYFQDEGSLTKKLCEQDSTFDGVTDKPILDCCACGTAKYRLTFYGNWSEKTHPKDYPRRANHWSAIIGGSHSKNYVLWEYGGYASEGVKQVAELGSPVKMEEEIRQQSDEVLTVIKAKAQWPAWQPLNVRAAPSAEFSVDRTRHLMSFLTMMGPSPDWNVGLSAEDLCTKECGWVQKVVQDLIPWDAGTDSGVTYESPNKPTIPQEKIRPLTSLDHPQSPFYDPEGGSITQVARVVIERIARKGEQCNIVPDNVDDIVADLAPEEKDEDDTPETCIYSNWSPWSACSSSTCDKGKRMRQRMLKAQLDLSVPCPDTQDFQPCMGPGCSDEDGPTCTMSEWITWSPCSISCGMGMRSRERYVKQFPEDGSVCTLPTEETEKCTVNEECSPSSCLMTEWGEWDEYSATCGMGMKKRHRMIKMNPADGSMCKAETSQAEKRMMPECHTIPCLLSPWSEWSDCSVTCGKGMRTRQRMLKSLAELGDCNEDLEQVEKCMLPECPIDCELTEWSQWSECNKSCGKGHVIRTRMIQMEPQFGGAPCPETVQRKKCRIRKCLRNPSIQKLRWREARESRRSEQLKEESEGEQFPGCRMRPWTAWSECTKLCGGGIQERYMTVKKRFKSSQFTSCKDKKEIRACNVHPC

SEQ ID NO: 3 - DNA, 1518 bp, Homo sapiens
ttgggggccgcgaagatgaggctgtccccggcgcccctgaagctgagccggactccggcactgctggccctggcgctgcccctggccgcggcgctggccttctccgacgagaccctggacaaagtgcccaagtcagagggctactgcagccgtatcctgcgcgcccagggcacgcggcgcgagggctacaccgagttcagcctccgcgtggagggcgaccccgacttctacaagccgggaaccagctaccgcgtaacactttcagctgctcctccctcctacttcagaggattcacattaattgccctcagagagaacagagagggtgataaggaagaagaccatgctgggaccttccagatcatagacgaagaagaaactcagtttatgagcaattgccctgttgcagtcactgaaagcactccacggaggaggacccggatccaggtgttttggatagcaccaccagcgggaacaggctgcgtgattctgaaggccagcatcgtacaaaaacgcattatttattttcaagatgagggctctctgaccaagaaactttgtgaacaagattccacatttgatggggtgactgacaaacccatcttagactgctgtgcctgcggaactgccaagtacagactcacattttatgggaattggtccgagaagacacacccaaaggattaccctcgtcgggccaaccactggtctgcgatcatcggaggatcccactccaagaattatgtactgtgggaatatggaggatatgccagcgaaggcgtcaaacaagttgcagaattgggctcacccgtgaaaatggaggaagaaattcgacaacagagtgatgaggtcctcaccgtcatcaaagccaaagcccagtggccagcctggcagcctctcaacgtgagagcagcaccttcagctgaattttccgtggacagaacgcgccatttaatgtccttcctgaccatgatgggccctagtcccgactggaacgtaggcttatctgcagaagatctgtgcaccaaggaatgtggctgggtccagaaggtggtgcaagacctgattccctgggacgctggcaccgacagcggggtgacctatgagtcacccaacaaacccaccattccccaggagaaaatccggcccctgaccagcctggaccatcctcagagtcctttctatgacccagagggtgggtccatcactcaagtagccagagttgtcatcgagagaatcgcacggaagggtgaacaatgcaatattgtacctgacaatgtcgatgatattgtagctgacctggctccagaagagaaagatgaagatgacacccctgaaacctgcatctactccaactggtccccatggtccgcctgcagctcctccacctgtgacaaaggcaagaggatgcgacagcgcatgctgaaagcacagctggacctcagcgtcccctgccctgacacccaggacttccagccctgcatgggccctggctgcagtgacgaagacggccccacc

SEQ ID NO: 4 - protein, 501 aa, Homo sapiens
MRLSPAPLKLSRTPALLALALPLAAALAFSDETLDKVPKSEGYCSRILRAQGTRREGYTEFSLRVEGDPDFYKPGTSYRVTLSAAPPSYFRGFTLIALRENREGDKEEDHAGTFQIIDEEETQFMSNCPVAVTESTPRRRTRIQVFWIAPPAGTGCVILKASIVQKRIIYFQDEGSLTKKLCEQDSTFDGVTDKPILDCCACGTAKYRLTFYGNWSEKTHPKDYPRRANHWSAIIGGSHSKNYVLWEYGGYASEGVKQVAELGSPVKMEEEIRQQSDEVLTVIKAKAQWPAWQPLNVRAAPSAEFSVDRTRHLMSFLTMMGPSPDWNVGLSAEDLCTKECGWVQKVVQDLIPWDAGTDSGVTYESPNKPTIPQEKIRPLTSLDHPQSPFYDPEGGSITQVARVVIERIARKGEQCNIVPDNVDDIVADLAPEEKDEDDTPETCIYSNWSPWSACSSSTCDKGKRMRQRMLKAQLDLSVPCPDTQDFQPCMGPGCSDEDGPT

SEQ ID NO: 5 - DNA, 1857 bp, Homo sapiens
ttgggggccgcgaagatgaggctgtccccggcgcccctgaagctgagccggactccggcactgctggccctggcgctgcccctggccgcggcgctggccttctccgacgagaccctggacaaagtgcccaagtcagagggctactgcagccgtatcctgcgcgcccagggcacgcggcgcgagggctacaccgagttcagcctccgcgtggagggcgaccccgacttctacaagccgggaaccagctaccgcgtaacactttcagctgctcctccctcctacttcagaggattcacattaattgccctcagagagaacagagagggtgataaggaagaagaccatgctgggaccttccagatcatagacgaagaagaaactcagtttatgagcaattgccctgttgcagtcactgaaagcactccacggaggaggacccggatccaggtgttttggatagcaccaccagcgggaacaggctgcgtgattctgaaggccagcatcgtacaaaaacgcattatttattttcaagatgagggctctctgaccaagaaactttgtgaacaagattccacatttgatggggtgactgacaaacccatcttagactgctgtgcctgcggaactgccaagtacagactcacattttatgggaattggtccgagaagacacacccaaaggattaccctcgtcgggccaaccactggtctgcgatcatcggaggatcccactccaagaattatgtactgtgggaatatggaggatatgccagcgaaggcgtcaaacaagttgcagaattgggctcacccgtgaaaatggaggaagaaattcgacaacagagtgatgaggtcctcaccgtcatcaaagccaaagcccagtggccagcctggcagcctctcaacgtgagagcagcaccttcagctgaattttccgtggacagaacgcgccatttaatgtccttcctgaccatgatgggccctagtcccgactggaacgtaggcttatctgcagaagatctgtgcaccaaggaatgtggctgggtccagaaggtggtgcaagacctgattccctgggacgctggcaccgacagcggggtgacctatgagtcacccaacaaacccaccattccccaggagaaaatccggcccctgaccagcctggaccatcctcagagtcctttctatgacccagagggtgggtccatcactcaagtagccagagttgtcatcgagagaatcgcacggaagggtgaacaatgcaatattgtacctgacaatgtcgatgatattgtagctgacctggctccagaagagaaagatgaagatgacacccctgaaacctgcatctactccaactggtccccatggtccgcctgcagctcctccacctgtgacaaaggcaagaggatgcgacagcgcatgctgaaagcacagctggacctcagcgtcccctgccctgacacccaggacttccagccctgcatgggccctggctgcagtgacgaagacggccccacctgcaccatgtccgagtggatcacctggtcgccctgcagcatctcctgcggcatgggcatgaggtcccgggagaggtatgtgaagcagttcccggaggacggctccgtgtgcacgctgcccactgaggaaacggagaagtgcacggtcaacgaggagtgctctcccagcagctgcctgatgaccgagtggggcgagtgggacgagtacagcgccacctgcggcatgggcatgaagaagcggcaccgcatgatcaagatgaaccccgcagatggctccatgtgcaaagccgagacatcacaggcagagaagcgcatgatgccagagtgccacaccatccca

SEQ ID NO: 6 - protein, 614 aa, Homo sapiens
MRLSPAPLKLSRTPALLALALPLAAALAFSDETLDKVPKSEGYCSRILRAQGTRREGYTEFSLRVEGDPDFYKPGTSYRVTLSAAPPSYFRGFTLIALRENREGDKEEDHAGTFQIIDEEETQFMSNCPVAVTESTPRRRTRIQVFWIAPPAGTGCVILKASIVQKRIIYFQDEGSLTKKLCEQDSTFDGVTDKPILDCCACGTAKYRLTFYGNWSEKTHPKDYPRRANHWSAIIGGSHSKNYVLWEYGGYASEGVKQVAELGSPVKMEEEIRQQSDEVLTVIKAKAQWPAWQPLNVRAAPSAEFSVDRTRHLMSFLTMMGPSPDWNVGLSAEDLCTKECGWVQKVVQDLIPWDAGTDSGVTYESPNKPTIPQEKIRPLTSLDHPQSPFYDPEGGSITQVARVVIERIARKGEQCNIVPDNVDDIVADLAPEEKDEDDTPETCIYSNWSPWSACSSSTCDKGKRMRQRMLKAQLDLSVPCPDTQDFQPCMGPGCSDEDGPTCTMSEWITWSPCSISCGMGMRSRERYVKQFPEDGSVCTLPTEETEKCTVNEECSPSSCLMTEWGEWDEYSATCGMGMKKRHRMIKMNPADGSMCKAETSQAEKRMMPECHTIP

SEQ ID NO: 7 - DNA, 2277 bp, Homo sapiens
ttgggggccgcgaagatgaggctgtccccggcgcccctgaagctgagccggactccggcactgctggccctggcgctgcccctggccgcggcgctggccttctccgacgagaccctggacaaagtgcccaagtcagagggctactgcagccgtatcctgcgcgcccagggcacgcggcgcgagggctacaccgagttcagcctccgcgtggagggcgaccccgacttctacaagccgggaaccagctaccgcgtaacactttcagctgctcctccctcctacttcagaggattcacattaattgccctcagagagaacagagagggtgataaggaagaagaccatgctgggaccttccagatcatagacgaagaagaaactcagtttatgagcaattgccctgttgcagtcactgaaagcactccacggaggaggacccggatccaggtgttttggatagcaccaccagcgggaacaggctgcgtgattctgaaggccagcatcgtacaaaaacgcattatttattttcaagatgagggctctctgaccaagaaactttgtgaacaagattccacatttgatggggtgactgacaaacccatcttagactgctgtgcctgcggaactgccaagtacagactcacattttatgggaattggtccgagaagacacacccaaaggattaccctcgtcgggccaaccactggtctgcgatcatcggaggatcccactccaagaattatgtactgtgggaatatggaggatatgccagcgaaggcgtcaaacaagttgcagaattgggctcacccgtgaaaatggaggaagaaattcgacaacagagtgatgaggtcctcaccgtcatcaaagccaaagcccagtggccagcctggcagcctctcaacgtgagagcagcaccttcagctgaattttccgtggacagaacgcgccatttaatgtccttcctgaccatgatgggccctagtcccgactggaacgtaggcttatctgcagaagatctgtgcaccaaggaatgtggctgggtccagaaggtggtgcaagacctgattccctgggacgctggcaccgacagcggggtgacctatgagtcacccaacaaacccaccattccccaggagaaaatccggcccctgaccagcctggaccatcctcagagtcctttctatgacccagagggtgggtccatcactcaagtagccagagttgtcatcgagagaatcgcacggaagggtgaacaatgcaatattgtacctgacaatgtcgatgatattgtagctgacctggctccagaagagaaagatgaagatgacacccctgaaacctgcatctactccaactggtccccatggtccgcctgcagctcctccacctgtgacaaaggcaagaggatgcgacagcgcatgctgaaagcacagctggacctcagcgtcccctgccctgacacccaggacttccagccctgcatgggccctggctgcagtgacgaagacggccccacctgcaccatgtccgagtggatcacctggtcgccctgcagcatctcctgcggcatgggcatgaggtcccgggagaggtatgtgaagcagttcccggaggacggctccgtgtgcacgctgcccactgaggaaacggagaagtgcacggtcaacgaggagtgctctcccagcagctgcctgatgaccgagtggggcgagtgggacgagtacagcgccacctgcggcatgggcatgaagaagcggcaccgcatgatcaagatgaaccccgcagatggctccatgtgcaaagccgagacatcacaggcagagaagcgcatgatgccagagtgccacaccatcccatgcttgctgtccccatggtccgagtggagtgactgcagcgtgacctgcgggaagggcatgcgaacccgacagcggatgctcaagtctctggcagaacttggagactgcaatgaggatctggagcaggtggagaagtgcatgctccctgaatgccccattgactgtgagctcaccgagtggtcccagtggtcggaatgtaacaagtcatgtgggaaaggccacgtgattcgaacccggatgatccaaatggagcctcagtttggaggtgcaccctgcccagagactgtgcagcgaaaaaagtgccgcatccgaaaatgccttcgaaatccatccatccaaaagctacgctggagggaggcccgagagagccggcggagtgagcagctgaaggaagagtctgaaggggagcagttcccaggt

SEQ ID NO: 8 - protein, 754 aa, Homo sapiens
MRLSPAPLKLSRTPALLALALPLAAALAFSDETLDKVPKSEGYCSRILRAQGTRREGYTEFSLRVEGDPDFYKPGTSYRVTLSAAPPSYFRGFTLIALRENREGDKEEDHAGTFQIIDEEETQFMSNCPVAVTESTPRRRTRIQVFWIAPPAGTGCVILKASIVQKRIIYFQDEGSLTKKLCEQDSTFDGVTDKPILDCCACGTAKYRLTFYGNWSEKTHPKDYPRRANHWSAIIGGSHSKNYVLWEYGGYASEGVKQVAELGSPVKMEEEIRQQSDEVLTVIKAKAQWPAWQPLNVRAAPSAEFSVDRTRHLMSFLTMMGPSPDWNVGLSAEDLCTKECGWVQKVVQDLIPWDAGTDSGVTYESPNKPTIPQEKIRPLTSLDHPQSPFYDPEGGSITQVARVVIERIARKGEQCNIVPDNVDDIVADLAPEEKDEDDTPETCIYSNWSPWSACSSSTCDKGKRMRQRMLKAQLDLSVPCPDTQDFQPCMGPGCSDEDGPTCTMSEWITWSPCSISCGMGMRSRERYVKQFPEDGSVCTLPTEETEKCTVNEECSPSSCLMTEWGEWDEYSATCGMGMKKRHRMIKMNPADGSMCKAETSQAEKRMMPECHTIPCLLSPWSEWSDCSVTCGKGMRTRQRMLKSLAELGDCNEDLEQVEKCMLPECPIDCELTEWSQWSECNKSCGKGHVIRTRMIQMEPQFGGAPCPETVQRKKCRIRKCLRNPSIQKLRWREARESRRSEQLKEESEGEQFPG

SEQ ID NO: 9 - DNA, 690 bp, Homo sapiens
ttgggggccgcgaagatgaggctgtccccggcgcccctgaagctgagccggactccggcactgctggccctggcgctgcccctggccgcggcgctggccttctccgacgagaccctggacaaagtgcccaagtcagagggctactccagccgtatcctgcgcgcccagggcacgcggcgcgagggctacaccgagttcagcctccgcgtggagggcgaccccgacttctacaagccgggaaccagctaccgcgtaacactttcagctgctcctccctcctacttcagaggattcacattaattgccctcagagagaacagagagggtgataaggaagaagaccatgctgggaccttccagatcatagacgaagaagaaactcagtttatgagcaattgccctgttgcagtcactgaaagcactccacggaggaggacccggatccaggtgttttggatagcaccaccagcgggaacaggctgcgtgattctgaaggccagcatcgtacaaaaacgcattatttattttcaagatgagggctctctgaccaagaaactttgtgaacaagattccacatttgatggggtgactgacaaacccatcttagactgctgtgcctgcggaactgccaagtacagactcacattttatgggaattggtccgagaagacacacccaaaggattaccct

SEQ ID NO: 10 - protein, 225 aa, Homo sapiens
MRLSPAPLKLSRTPALLALALPLAAALAFSDETLDKVPKSEGYCSRILRAQGTRREGYTEFSLRVEGDPDFYKPGTSYRVTLSAAPPSYFRGFTLIALRENREGDKEEDHAGTFQIIDEEETQFMSNCPVAVTESTPRRRTRIQVFWIAPPAGTGCVILKASIVQKRIIYFQDEGSLTKKLCEQDSTFDGVTDKPILDCCACGTAKYRLTFYGNWSEKTHPKDYP

SEQ ID NO: 11 - DNA, 1341 bp, Homo sapiens
ttgggggccgcgaagatgaggctgtccccggcgcccctgaagctgagccggactccggcactgctggccctggcgctgcccctggccgcggcgctggccttctccgacgagaccctggacaaagtgcccaagtcagagggctactgcagccgtatcctgcgcgcccagggcacgcggcgcgagggctacaccgagttcagcctccgcgtggagggcgaccccgacttctacaagccgggaaccagctaccgcgtaacactttcagctgctcctccctcctacttcagaggattcacattaattgccctcagagagaacagagagggtgataaggaagaagaccatgctgggaccttccagatcatagacgaagaagaaactcagtttatgagcaattgccctgttgcagtcactgaaagcactccacggaggaggacccggatccaggtgttttggatagcaccaccagcgggaacaggctgcgtgattctgaaggccagcatcgtacaaaaacgcattatttattttcaagatgagggctctctgaccaagaaactttgtgaacaagattccacatttgatggggtgactgacaaacccatcttagactgctgtgcctgcggaactgccaagtacagactcacattttatgggaattggtccgagaagacacacccaaaggattaccctcgtcgggccaaccactggtctgcgatcatcggaggatcccactccaagaattatgtactgtgggaatatggaggatatgccagcgaaggcgtcaaacaagttgcagaattgggctcacccgtgaaaatggaggaagaaattcgacaacagagtgatgaggtcctcaccgtcatcaaagccaaagcccagtggccagcctggcagcctctcaacgtgagagcagcaccttcagctgaattttccgtggacagaacgcgccatttaatgtccttcctgaccatgatgggccctagtcccgactggaacgtaggcttatctgcagaagatctgtgcaccaaggaatgtggctgggtccagaaggtggtgcaagacctgattccctgggacgctggcaccgacagcggggtgacctatgagtcacccaacaaacccaccattccccaggagaaaatccggcccctgaccagcctggaccatcctcagagtcctttctatgacccagagggtgggtccatcactcaagtagccagagttgtcatcgagagaatcgcacggaagggtgaacaatgcaatattgtacctgacaatgtcgatgatattgtagctgacctggctccagaagagaaagatgaagatgacacccctgaaacc

SEQ ID NO: 12 - protein, 442 aa, Homo sapiens
MRLSPAPLKLSRTPALLALALPLAAALAFSDETLDKVPKSEGYCSRILRAQGTRREGYTEFSLRVEGDPDFYKPGTSYRVTLSAAPPSYFRGFTLIALRENREGDKEEDHAGTFQIIDEEETQFMSNCPVAVTESTPRRRTRIQVFWIAPPAGTGCVILKASIVQKRIIYFQDEGSLTKKLCEQDSTFDGVTDKPILDCCACGTAKYRLTFYGNWSEKTHPKDYPRRANHWSAIIGGSHSKNYVLWEYGGYASEGVKQVAELGSPVKMEEEIRQQSDEVLTVIKAKAQWPAWQPLNVRAAPSAEFSVDRTRHLMSFLTMMGPSPDWNVGLSAEDLCTKECGWVQKVVQDLIPWDAGTDSGVTYESPNKPTIPQEKIRPLTSLDHPQSPFYDPEGGSITQVARVVIERIARKGEQCNIVPDNVDDIVADLAPEEKDEDDTPET